(19) United States
(12) Patent Application Publication
(10) Pub. No.: US 2013/0136688 A1
(43) Pub. Date: May 30, 2013

(54) Nanoparticles for Targeted Delivery of Active Agents to the Lung

(71) Applicants: Jürgen Borlak, Lehrte/OT Immensen (DE); Shimon Benita, Tel Aviv (IL); Nir Debotton, Tel Aviv (IL); Nour Karra, Tel Aviv (IL); Oshrat Harush-Frenkel, Modiin (IL)

(72) Inventors: Jürgen Borlak, Lehrte/OT Immensen (DE); Shimon Benita, Tel Aviv (IL); Nir Debotton, Tel Aviv (IL); Nour Karra, Tel Aviv (IL); Oshrat Harush-Frenkel, Modiin (IL)

(21) Appl. No.: 13/687,288

(22) Filed: Nov. 28, 2012

Related U.S. Application Data
(62) Division of application No. 12/889,739, filed on Sep. 24, 2010, now abandoned.
Application No. 12/889,739 is a continuation of application No. PCT/EP2009/002513, filed on Mar. 31, 2009.

(30) Foreign Application Priority Data
Mar. 31, 2008 (EP) 08075267.8

(51) Int. Cl. A61K 9/14

(52) U.S. Cl. CPC A61K 9/146
USPC 424/1.13; 424/489; 424/178.1; 424/1.37

(57) ABSTRACT

The present invention concerns a delivery system administered to the lung preferably by inhalation comprising a polymer-based nanoparticle; and a linker comprising a first portion non-covalently anchored to said nanoparticle, wherein at least part of said first portion comprises a hydrophobic/lipophilic segment embedded in said nanoparticle; and a second portion comprising a coupling group, preferably a maleimide compound, exposed at the outer surface of said nanoparticle. In accordance with one embodiment, the delivery system comprises one or more targeting agents, each covalently bound to said coupling group, preferably maleimide compound, and is administered as an aerosol in the therapy or diagnosis of lung cancer or bronchial dysplasia. In accordance with yet another embodiment, the delivery system comprises a drug and/or a radiopharmaceutical and/or a contrasting agent. A specific example for a linker in accordance with the invention is octadecyl-4-(maleimideomethyl)cyclohexane-carboxylic amide (OMCCA).

### CROSS-REFERENCE TO RELATED APPLICATIONS

This application is a divisional application of 12/889,739, filed Sep. 24, 2010 which is a continuation of PCT International Application No. PCT/EP2009/002513, filed on Mar. 31, 2009, and claiming priority to European Application No. 08075267.8, filed on Mar. 31, 2008. Both of those applications are incorporated by reference herein.

### BACKGROUND OF THE INVENTION

1. Field of the Invention

Embodiments of the invention relate to polymer-based nanoparticles for use as local delivery vehicles for the lung by intravenous application and/or by inhalation.

2. Background of the Related Art

Nanoparticles (NPs) have shown great potential as carrier systems for an increasing number of active molecules including hydrophobic potent cytotoxic drugs. Although capable of enhanced accumulation in the target tissue compared to plain particles, the NPs cannot provide targeting unless specific ligands such as monoclonal antibodies (MAbs) are attached to them. Coupling of MAbs to NPs is attained by covalent binding of the antibody molecule to the particle surface. These immunonanoparticles (immunoNPS) should ensure the specific recognition of the antigen site by the antibody and the release of paclitaxel palmitate, a lipophilic pro-drug of paclitaxel by the colloidal carrier close to the inaccessible pathological lung target tissues over-expressing such tumor antigens.

Aerosol therapy using particulate drug carrier systems is becoming a popular method to deliver therapeutic compounds either locally or systemically [12]. Pulmonary delivery using metered dose inhaler systems for aerosols or powders may contain nanostructures such as NPs. Research into lung delivery is driven by the potential for successful protein and peptide drug delivery by this route. Pulmonary drug delivery offers local targeting for the treatment of respiratory diseases.

However, the success of pulmonary delivery of protein drugs is diminished by proteases and macrophages in the lung, which reduce their overall bioavailability, and by the barrier between capillary blood and alveolar air. Targeting mechanisms can make therefore, advantage of both, drug formulation and route of administration, and can be either passive or active. An example of passive targeting is the preferential accumulation of chemotherapeutic agents in solid tumors as a result of the differences in the vascularization of the tumor tissue compared with healthy tissue.

The interest in biodegradable particles with diameters in the nanometer range (preferably <250 nm) for pulmonary delivery has grown.

It should be emphasized that up to now, to the best of our knowledge immunoNPs were not administered by aerosol therapy. It will be interesting to assess the biofate of immunoNPs as compared to NPs. However, the safety and toxicology of these systems in the lung may be problematic in part because of their extensive surface area. Different-sized “inert” particles composed of polystyrene and TiO₂ induced a surface area-dependent pulmonary-inflammatory response following their introduction to the lung.

Thus, there is a great need for a system appropriate for the safe and easy local lung delivery of active agents, in particular in the therapy and diagnosis of lung cancer. The aim of the present invention is therefore to make available safe and easy means for local lung delivery, in particular for the therapy and diagnosis of cancer or dysplasia.

To this end, the implementation of the actions and embodiments as described in the claims provides appropriate means to fulfill these demands in a satisfying manner.

In the present study there is made use of PLA/PLGA, a polymer approved for injection by the FDA for the formation of NPs and immunoNPs due to their safe biocompatible and biodegradable properties. The advantages of coupling NPs containing lipophilic cytotoxic drugs with MAbs followed by local delivery to the lungs include: direct delivery to lungs, prolonged residence time in the target tissue; continuous release of significant potent drug doses at tumor sites and better cytotoxic drug internalization in tumors allowing improved efficacy.

It should be emphasized that no published work showed the local delivery of immunonanoparticles by inhalation directly to the lungs.

### BRIEF SUMMARY OF THE INVENTION

Embodiments of the invention are based on the surprising finding, that the development of a simple approach for associating targeting agent, such as antibodies, to polymer-based nanoparticles (preferably those comprising a therapeutically active agent), which does not require a priori chemical binding of the targeting agent to the particle-forming polymer combined with an active agent, results in products allowing a fast, safe and easy delivery of the active agent to targeted sites, e.g. tissues or cells affected by cancer or dysplasia, in the lung. This was achieved by the use of a molecular linker having a lipophilic portion which non-covalently anchors to the particle's polymeric matrix and a second portion comprising a coupling group, preferably a maleimide compound, to which it is possible in a subsequent step to bind the targeting agent. This novel approach eliminates the need to tailor for each different targeting agent a different nanoparticle composition, and enables to form a “universal” nanoparticle-linker (with an active agent such as a cytotoxic agent), which can be used to prepare different targeted systems, for enhanced local lung delivery, simply by binding to the linker different targeting agents (ligands) according to needs.

Thus, embodiments of the present invention concern a delivery system comprising:

(i) a polymer-based nanoparticle;
(ii) a linker comprising a first portion non-covalently anchored to said nanoparticle, wherein at least part of said first portion comprises a lipophilic segment embedded in said nanoparticle; and a second portion comprising a coupling group exposed at the outer surface of said nanoparticle to which a ligand (targeting moiety) has been covalently coupled; and
(iii) an active agent selected from the group consisting of: a drug, a radiopharmaceutical and a contrasting agent;

in local lung delivery, in particular by intravenous application and/or by pulmonary administration, preferably by inhalation.

The coupling group is preferably selected from the group consisting of Maleimide, NHS-ester, Carbodiimide, Hydrazide, PFP-ester, Hydroxymethyl Phosphine, Psoralen, Imidoester, Pyridyl Disulfide, Isocyanate, Vinyl Sulfone, alpha-haloacetyls, Aryl Azide, Diazirine, and Benzophenone.

Thus, according to a first of its aspects, the present invention provides a delivery system for local lung delivery by inhalation comprising:

(i) a polymer-based nanoparticle;
(ii) a linker comprising a first portion non-covalently anchored to said nanoparticle, wherein at least part of said first portion comprises a hydrophobic segment embedded in said nanoparticle; and a second portion comprising a maleimide compound exposed at the outer surface of said nanoparticle;
(iii) a drug; and
(iv) a ligand (targeting moiety).

The nanoparticle preferably comprises an active agent carried by the particle, such as a drug, a contrasting agent and combinations of same, embedded, conjugated, impregnated, or encapsulated in said particle, or adsorbed at the surface of the particle.

The above nanoparticle-linker can be used in subsequent production of the final targeted product, as the linker is suitable for covalent binding with a targeting agent.

According to one preferred embodiment, the nanoparticle comprises one or more targeting agents (targeting moieties) each covalently bound to said maleimide compound.

Embodiments also provide a composition comprising the delivery system of the invention. In accordance with one embodiment, the composition comprises a pharmaceutically acceptable carrier. In accordance with some other embodiments, the composition comprises an active agent carried by said nanoparticle.

Embodiments also provide a method for treating or preventing a disease or disorder related to the lungs, the method comprises providing a subject in need, an amount of the delivery system of the invention by inhalation, the amount being effective to treat or prevent said disease or disorder.

Yet further, embodiments provide a method of diagnosis in a subject's body a target cell or target tissue, the method comprising:

(a) providing said subject with the delivery system of the invention and carrying a contrasting agent wherein the nanoparticles are associated with one or more targeting agents effective to target said delivery system to said target cell or target tissue in the lung;
(b) imaging said contrasting agent in said body.
Furthermore, the invention provides a method of producing the delivery system for local lung delivery.

### BRIEF DESCRIPTION OF THE FIGURES

FIGS. 1A-1C are schematic illustrations of a delivery particle according to the invention, in which a linker (OMCCA) has a first portion anchored in the particle, and a second portion (maleimide) exposed at the surface of the particle and associated to an antibody (Y) (FIG. 1A); the delivery particle may further comprise portions of the polymer modified with polyethylene glycol (FIG. 1B), and may also carry a drug embedded in the polymeric matrix (FIG. 1C).

FIG. 2 is a three dimensional bar graph showing zeta potential measurements for non-conjugated particles (blank), trastuzumab-conjugated particles (immunoNPs), trastuzumab-conjugated and drug loaded particles (immuno DCTX NPs).

FIGS. 3A-3B shows transmission electron microscopy images of antibody-conjugated nanoparticles according to the invention, using 12 nm gold labeled goat anti-human IgG, at two scales, 200 nm (FIG. 3A) and 100 nm (FIG. 3B).

FIG. 4 shows animal weight change following 3 and 14 days recovery in mice instilled with NPs and immunonanoparticles over 5 consecutive days.

FIG. 5A-5D show total BAL cells following 3 and 14 days recovery in mice instilled with NPs and immunonanoparticles over 5 consecutive days (FIG. 5A), total BAL macrophages (FIG. 5B), total BAL lymphocytes (FIG. 5C) and total BAL neutrophils (FIG. 5D).

FIG. 6: Scoring of macrophages, congestion and inflammation within both lungs of mice installed with different formulations following 3 (FIG. 6A) and 14 (FIG. 6B) days recovery.

FIG. 7 are images of lung tissues from mice instilled with different formulations and scarified on the 8 (FIG. 7A) and 19 (FIG. 7B) days of experiment

FIG. 8: Weight gain of mice after eighth (black bars) and nineteenth (grey bars) experiment days compared to the weight on the first day of experiment.

FIG. 9: Total BAL cell counts (FIG. 9A), Macrophage cell count within BAL (FIG. 9B), Lymphocyte cell count within BAL (FIG. 9C), granulocyte cell count within BAL (FIG. 9D). Results are for both eighth (grey bars) and nineteenth (black bars) experiment days.

FIG. 10: Macrophages, congestion and inflammation scoring within both lungs of mice installed with different formulations on the eighth (FIG. 10A) and nineteenth (FIG. 10B) experiment days. Results presented as means±SEM, n=3. Ranking index of macrophages was 1—few macrophages in several alveoli, 2—few macrophages in 10-20% of the alveoli, 3—grouped (2-3) macrophages in 10-20% of the alveoli, 4—grouped (2-3) macrophages in above 30% of the alveoli. Inflammation index was 1—no inflammation, 2—very few foci of the lymphatic aggregates (probably reactive BALT-broncho-associated lymapthatic tissue), 3—few foci of chronic infiltrates dispersed in the interstitial tissue of the lung, 4—slight increase in number of foci of chronic infiltrates dispersed in the interstitial tissue of the lung. Indexing of congestion was as follows 1—no congestion, 2—foci of moderate engorgement of capillaries in the alveolar septi, 3—foci of moderate engorgement of capillaries in the alveolar septi accompanied by slight edema and thickening of the involved septi.

FIG. 11: Lung tissues from mice instilled with different formulations and scarified on the eighth (a-e) and nineteenth (f-j) days of experiment. Magnifications of pictures presented are X 400. The pictures presented: a,f. control saline, b,g. PEG-PLA NPs, c,h. SA-PEG-PLA NPs, d,i. control EpCAM saline, e,j. EpCAM NPs.

FIG. 12: Representative pictures of EpCAM immunohistochemistry of mice lungs tissue sections instilled with control saline (A,B,a,b), control EpCAM saline (C,D,c,d) as well as EpCAM conjugated NPs (E,F,e,f) and sacrified on the eighth experiment day. The different treatments (A,a,C,c,E,e) were either incubated with primary EpCAM antibody and secondary rat anti-mouse antibody or with secondary rat anti-mouse antibody solely (B,b,D,d,F,f) as negative control. Original magnifications are ×10 (A,B,C,D) or ×40 (a,b,c,d).

FIG. 13: Representative pictures of EpCAM immunohistochemistry of mice lungs instilled with control EpCAM saline (A,B,a,b) and EpCAM conjugated NPs (C,D,c,d) and sacrified on the nineteenth experiment day. Positive control for EpCAM expression is depicted in colon tissue of mice colon (E,e,F,f). The different treatments (A,a,C,c,E,e) were either incubated with primary EpCAM antibody and secondary rat anti-mouse antibody or with secondary rat anti-mouse antibody solely (B,b,D,d,F,f) as negative control. Original magnifications are ×10 (A,B,C,D) or ×40(a,b,c,d).

FIG. 14: FIG. 14A and FIG. 14B show transgenic lung tumor bearing mice receiving formulations via an aerosol endotracheal administration.

FIG. 15: Body weight change within groups during study period.

FIG. 16: Seven-day differences in body weight change.

FIG. 17: Blood counts—monocytes.

FIG. 18: LDH/Total cell count in BAL (FIG. 18a); neutrophils in BAL (FIG. 18b).

FIG. 19: Macrophages in BAL

FIG. 20: Immunohistochemistry showing EpCAM's presence in the lungs.

FIG. 21: Organ distribution of 68Ga-labeled NOTA-chelated anti-EpCAM antibody (FIG. 21C) as well as DOTA (FIG. 21A) and DTPA-chelated nanoparticles (FIG. 21B).

### DETAILED DESCRIPTION OF THE INVENTION

The present invention is aimed to provide improvement of drug delivery therapy to the lung which is based on a novel one-step conjugation process of one or more targeting agents to drug-loaded nanoparticles. In particular, the invention enables the preparation of a universal nanoparticle linker (optionally in combination with a drug) that can be subsequently bound to a targeting agent of choice, so that there is no need to design a special nanoparticle for each different targeting agent. The design nanoparticles in accordance with the invention allow a better recognition of targeted cells exhibiting two surface membrane low antigen densities.

The present invention thus provides a product comprising
(i) a polymer-based nanoparticle,
(ii) a linker comprising a first portion non-covalently anchored to said nanoparticle, wherein at least part of said first portion comprises a lipophilic segment embedded in said nanoparticle; and a second portion comprising a coupling group exposed at the outer surface of said nanoparticle, wherein a ligand (targeting moiety) has been covalently coupled (via a covalent bond) to said coupling group, and
(iii) an active agent selected from the group consisting of a drug, a radiopharmaceutical and a contrasting agent

as a delivery system in local lung delivery, in particular by intravenous application and/or by inhalation.

Within the context of intravenous application, the delivery system is applied systemically to the body, but a local delivery to the lung is effected, thus allowing an easy and efficient drug targeting in the body.

Unexpectedly it has been found that inhalation of the delivery system according to the invention allows a particular safe and effective delivery of active agents to the lung. Thus, according to the invention the use of the delivery system for local lung delivery by inhalation is particularly preferred.

Preferably, the coupling group according to the invention is a chemical group selected from the group consisting of Maleimide, NHS-ester, Carbodiimide, Hydrazide, PFP-ester, Hydroxymethyl Phosphine, Psoralen, Imidoester, Pyridyl Disulfide, Isocyanate, Vinyl Sulfone, alpha-haloacetyls, Aryl Azide, Diazirine, and Benzophenone, and wherein the ligand has been covalently coupled to said chemical group via a chemical reaction.

The present invention thus provides delivery systems comprising a polymer based nanoparticle and a linker comprising a first portion non-covalently anchored to said nanoparticle, wherein at least part of said first portion comprises a hydrophobic segment embedded in said nanoparticle; and a second portion comprising a maleimide compound exposed at the outer surface of said nanoparticle.

Maleimides are a group of organic compounds with a 2,5-pyrroledione skeleton as depicted in general formula (I) hereinbelow.

Maleimides are used in a wide range of applications ranging from advanced composites in the aerospace industry to their use as reagents in synthesis. For example the aerospace industry requires materials with good thermal stability and a rigid backbone both of which are provided by bismaleimides. In some applications, various linkers such as polysiloxanes and phosphonates are conjugated to the bismaleimindes to strengthen polymers made therefrom, etc.

Maleimides may also be linked to polyethylene glycol chains which are often used as flexible linking molecules to attach proteins to surfaces. The double bond readily reacts with the thiol group found on cysteine to form a stable carbon-sulfur bond. Attaching the other end of the polyethylene chain to a bead or solid support allows for easy separation of protein from other molecules in solution, provided these molecules do not also possess thiol groups. In the context of the present invention, maleimide is conjugated to a linker to be incorporated non-covalently into a polymer based nanoparticle and the combination of the maleimide-linker with the nanoparticle provides a delivery system platform for various active agents.

The term “delivery system” which may be used herein interchangeably with the term “delivery nanoparticles” denotes physiologically acceptable, polymer-based nanoparticles which when associated with a linker, the particles have a diameter of 1 micrometer or less, preferably in the range of about 50-1000 nm, more preferably in the range of about 200-300 nm. While the nanoparticles preferably have a matrix structure formed from one or more polymers. Further, while the nanoparticles may be formed from substances other than a polymer, it is to be understood that the particles are essentially polymer-based or at least their outer surface is polymer-based. Thus, the term “nanoparticles” in the context of the invention excludes liposomes or emulsion forms.

The terms “polymer based particles”, “polymer based nanoparticles” or “particle forming polymer” as used herein denotes any biodegradable, and preferably biocompatible polymer capable of forming, under suitable conditions, nanoparticles A variety of biodegradable polymers is available in the art and such polymers are applicable in the present invention. Approved biodegradable, biocompatible and safe polymers largely used in nanoparticle preparations are described by Gilding D K et al. [Gilding D K et al. Polymer 20:1459-1464 (1979)].

Non-limiting examples of particle-forming biodegradable polymers are polyesters such as, without being limited thereto, polyhydroxybutyric acids, polyhydroxyvaleric acids; polycaprolactones; polyesteramides; polycyanoacrylates; poly(amino acids); polycarbonates; polyanhydrides; and mixtures of same.

Preferably, the polymer is selected from polylactic acid (polylactide), polylactide-polyglycolide, polyglycolide, poly(lactide-co-glycolide), polyethylene glycol-co-lactide (PEG-PLA) and mixtures of any of same.

A further component within the delivery system is the linker comprising a first portion non-covalently anchored to the nanoparticle and a second portion comprising a maleimide compound exposed at the outer surface of said nanoparticle. The first portion is configured such that at least part of same comprises a hydrophobic segment embedded in the nanoparticle's surface.

The term “anchor” as used herein denotes the penetration of at least part of the first portion of the linker through the particle's outer surface so as to obtain a stable association between the linker and the particle. The anchoring may be achieved by the incorporation of a moiety (herein termed “the anchor moiety”) at the first portion of the linker which has similar physical characteristics as the polymer. Those versed in chemistry will know how to select an anchor moiety to be compatible with the substance from which the particle is essentially made. For example, when using a hydrophobic polymer to form a particle matrix, a preferred selection of an anchor moiety is a hydrophilic and/or lipophilic moiety. In other words the anchor moiety should preferably be compatible with the polymer and eventually with the incorporated drug.

The association between the anchor moiety and the particle is preferably by mechanical fixation (e.g. by embedment) of the anchor to the polymer matrix. The mechanical fixation is obtained upon formation of the particles, when using the polymer in combination with the linker during polymer solidification process. Once the polymer solidifies in the form of particulates, it “captures” the anchor moiety of the linker to form the resulting delivery system of the invention.

The linker in the context of the present invention is an amphipathic molecule, i.e. a molecule having a hydrophobic/lipophilic portion (providing the anchor) and a maleimide compound forming part of the hydrophilic portion. It is noted that in the following whenever the term “lipophilic” is used, it may be understood interchangeably with the term hydrophilic, as long as the hydrophobic/lipophilic moiety is compatible with the polymer forming the nanoparticle. Thus, a lipophilic portion may equally refer to a hydrophilic portion. In accordance with some embodiments, the hydrophobic/lipophilic portion comprises a hydrocarbon or a lipid comprising at least 8 carbon atoms in the hydrocarbon backbone. An exemplary range is C₈-C₃₀ carbon atoms. The lipophilic moiety may be a saturated or unsaturated hydrocarbon, linear, branched and/or cyclic.

It is noted that the linker may have one or more anchors which may be incorporated in the nanoparticle's surface. For example, a double anchor may be achieved by the use of linker comprising 1,2-Distearoyl-sn-Glycero-3-Phosphoethanolamine-N-[Maleimide(Polyethylene Glycol)2000], shown in Table 1 below, which contains two lipophilic moieties. The linker has also a second portion to which a targeting agent (as disclosed below) binds. The binding of a targeting agent is preferably by covalent attachment, although non-covalent association may, at times, also be applicable. Covalent attachment is achieved by the inclusion in the hydrophilic portion of a chemically reactive group, in the instant invention, maleimide. Maleimide may form a stable thio-ether linkage with thiol groups of targeting agents.

According to some embodiments, the linker has the following general formula (I):

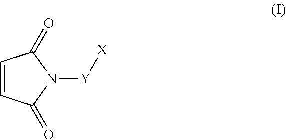

wherein

Y represents a heteroatom, a C₁-C₂₀ alkylene or alkenylene, a C₅-C₂₀ cycloalkylene or cycloalkenylene, C₆-C₂₀ alkylene-cycloalkykylene, wherein one of the carbon atoms in said alkylene or alkenylene may be replaced by a heteroatom;

X represents a carbonyl containing moiety selected from —C(O)—R₁, —C(O)—NH—R₁, —C(O)—O—C(O)—R₁, C(O)NH—R₂—R₁, or —C(O)—NH—R₂—C(O)—NH—R₁, wherein R₁ represents a hydrocarbon or a lipid comprising at least 8 carbons and R₂ represents a hydrophilic polymer.

In accordance with such embodiments, R₁ may represent a lipid; R₂ a hydrophilic polymer. According to one embodiment, the lipid is selected from mono or diacylglycerol, a phospholipid, a sphingolipid, a sphingophospholipid or a fatty acid.

It is noted that R₁ should be compatible with the polymer nanoparticle matrix and should be lipophilic. In accordance with this embodiment, Y may preferably represent an alkylene-cyclohexane.

The hydrophilic polymer may be any surface modifier polymer. Polymers typically used as surface modifiers include, without being limited thereto: polyethylene glycol (PEG), polysialic acid, polylactic (also termed polylactide), polyglycolic acid (also termed polyglycolide), apolylactic-polyglycolic acid, polyvinyl alcohol, polyvinylpyrrolidone, polymethoxazoline, polyethyloxazoline, polyhydroxyethyloxazoline, polyhydroxypropyloxazoline, polyaspartamide, polyhydroxypropyl methacrylamide, polymethacrylamide, polydimethylacrylamide, polyvinylmethylether, polyhydroxyethyl acrylate, derivatized celluloses such as hydroxymethylcellulose or hydroxyethylcellulose. The polymers may be employed as homopolymers or as block or random copolymers.

Preferably, the hydrophilic polymer is polyethylene glycol (PEG). The PEG moiety preferably has a molecular weight from about 750 Da to about 20,000 Da. More preferably, the molecular weight is from about 750 Da to about 12,000 Da and most preferably between about 2,000 Da to about 5,000 Da.

Preferably the polyethylene glycol is monomethoxypolyethylene glycol (monomethoxy or regular peg) Thus, a preferred lipopolymer utilized in accordance with the invention is stearylamine-monomethoxypoly(ethyleneglycol) (SA-mPEG).

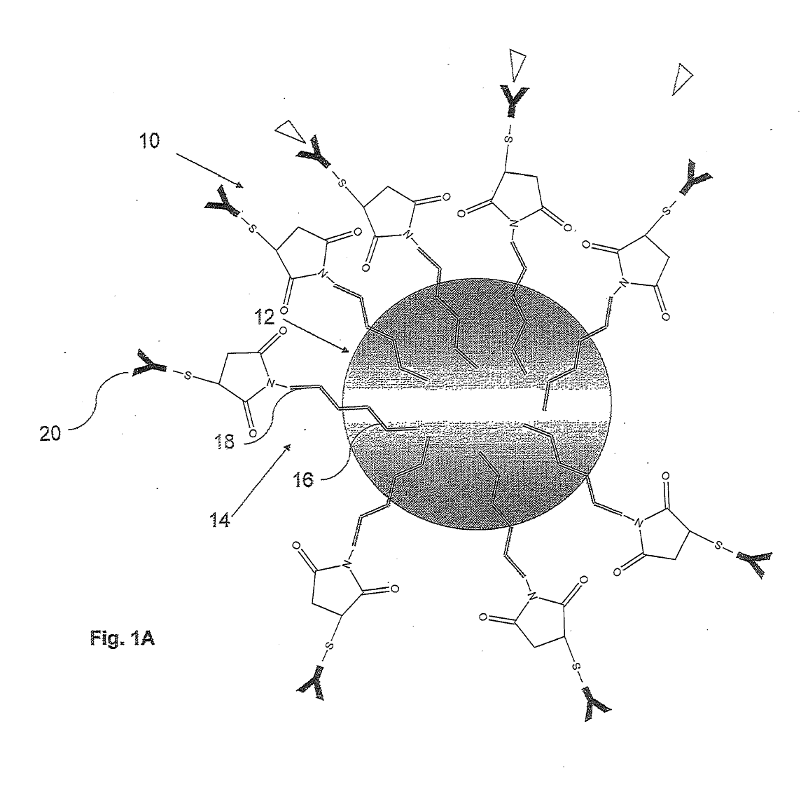

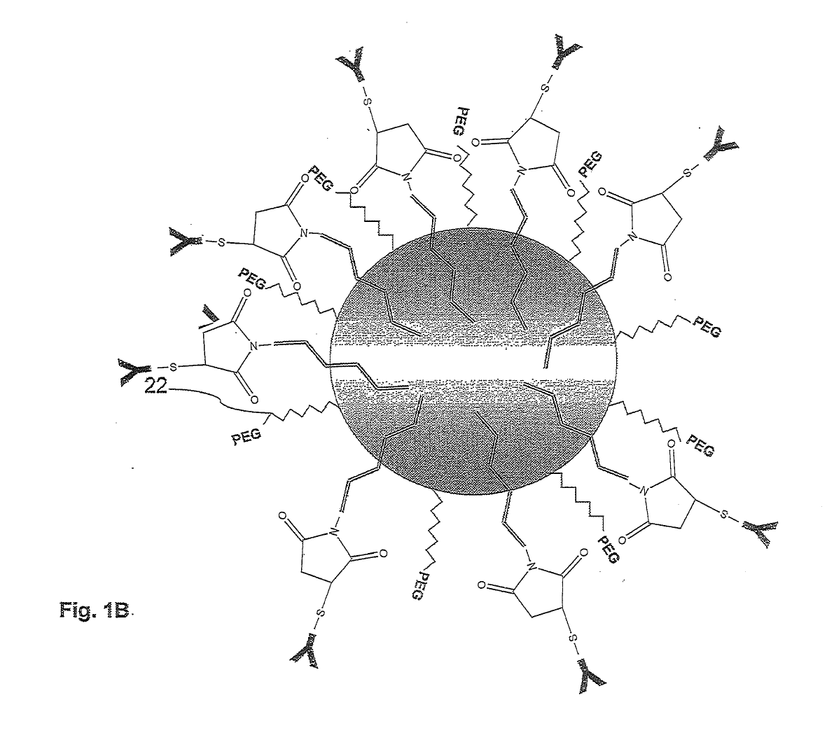

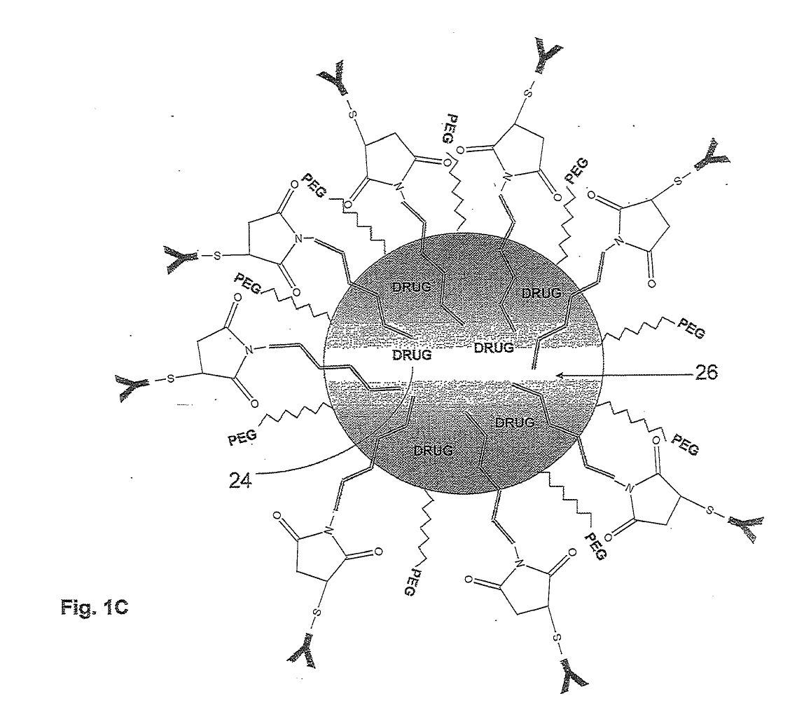

Alternatively, the hydrophilic polymer may be covalently to the polymer forming the particle, for example mPEG-polylactide, as schematically illustrated in FIG. 1B.

One particular embodiment of the invention concerns a compound of formula (I) wherein Y represents an alkylene-cycloalkykylene having the formula —CH₂—C₆H₁₀—; X represents a carbonyl containing moiety having the formula —C(O)—NH—R₁, wherein R₁ is a fatty acid.

The advantages of coupling NPs containing lipophilic cytotoxic drugs with MAbs followed by local delivery to the lungs include: direct delivery to lungs, prolonged residence time in the target tissue; continuous release of significant potent drug doses at tumor sites and better cytotoxic drug internalization in tumors allowing improved efficacy.

Another particular embodiment of the invention concerns a compound of formula (I) wherein the linker is selected from Octadecyl-4-(maleimidomethyl)cyclohexane-carboxylic amide (OMCCA); N-1 stearyl-maleimide (SM); succinimidyl oleate; 1,2-Distearoyl-sn-Glycero-3-Phosphoethanolamine-N-[Maleimide(Polyethylene Glycol)2000]; and mixtures thereof (Table 1):

The chemical structures of some applicable linkers are provided in the following Table 1.

TABLE 1
Chemical Names and Structures of Linkers (In each case, structure follows name)
Octadecyl-4-(maleimidomethyl)cyclohexane-carboxylic amide (OMCCA)
Octadecyl-4-(maleimidomethyl)cyclohexane-carboxylic amide 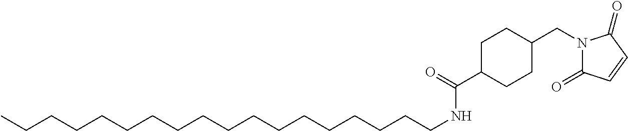 [SMILES: CCCCCCCCCCCCCCCCCCNC(=O)C1CCC(CN2C(=O)C=CC2=O)CC1]
Succinimidyl oleate

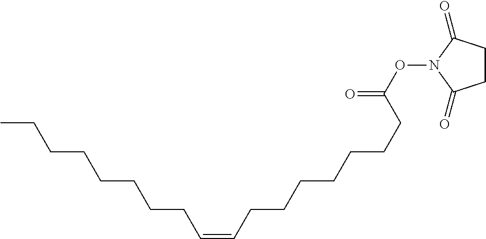 [SMILES: CCCCCCCC/C=C\CCCCCCCC(=O)ON1C(=O)CCC1=O]

Stearyl amine succinimidyl, 1,2-Distearoyl-sn-Glycero-3-Phosphoethanolamine-N-
[Maleimide(Polyethylene Glycol)2000]

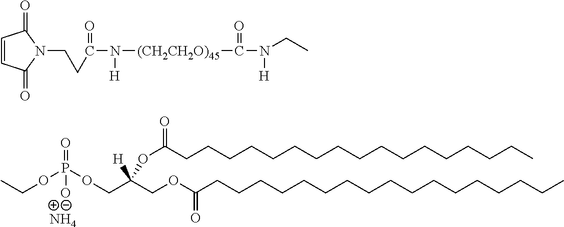

OMCCA, which is one preferred linker in accordance with the invention may be synthesized according to Scheme 1 below:

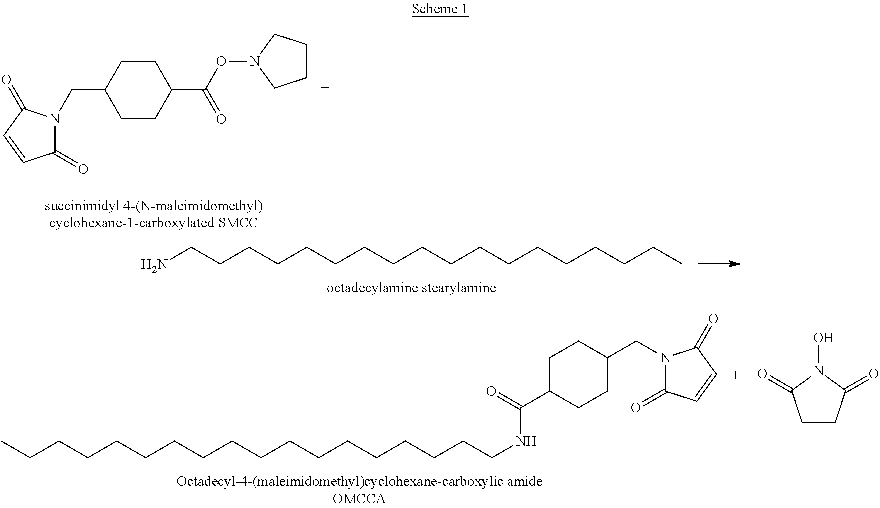

Succinimidyl oleate is commercially available from Sigma (Sigma Chemical, MO, USA; 1,2-Distearoyl-sn-glycero-3-phosphoethanolamine-N-[maleimide(polyethylene glycol)2000] is commercially available from AVANTI Polar Lipids inc, (Avanti Polar Lipids, Alabaster, Ala.). The delivery system of the invention may be provided in the form of a targeted delivery system, i.e. a delivery system attached to a targeting agent. At times, when the targeting agent is an antibody or a binding fragment thereof, the targeted delivery system of the invention may be referred as “Immunonanoparticles”.

The targeting agent may be regarded as one member of a binding couple the other member of the couple being the target on the cells, tissue to which the targeted delivery system of the invention should be selectively/preferably delivered. The term “binding couple” as used herein, signifies two substances, which are capable of specifically (affinity) binding to one another. Non-limiting examples of binding couples include biotin-avidin, antigen-antibody, receptor-ligand, oligonucleotide-complementary oligonucleotide, sugar-lectin, as known to those versed in the art.

The targeting agent may be a targeting polymer or oligomer. Non-limiting examples of polymers (and immunological functional fragments thereof) comprises amino acid-based polymers (e.g. antibodies, antigens, glycoproteins), nucleic acid-based polymers (e.g. immunostimulatory oligodeoxynucleodites (ODN), sense and antisense, interference RNA (iRNA) etc. or saccharide-based polymers, such as glycoproteins (e.g. lectins).

As noted above, also fragments of any of the above targeting may be used in accordance with the invention as long as they retain their specific binding properties to the target. When the targeting agent is an antibody (see definition below), the latter may be any one of the IgG, IgM, IgD, IgA, and IgG antibody, including polyclonal antibodies or monoclonal antibodies. Fragments of the antibodies may comprise the antigen-binding domain of an antibody, e.g. antibodies without the Fc portion, single chain antibodies, fragments consisting of essentially only the variable, antigen-binding domain of the antibody, etc.

In accordance with some embodiments, the targeting agent is a low molecular weight compound such as folic acid or thiamine. For example, thiamine may be bound to the linker anchored to the polymer based nanoparticle; and the thus formed nanoparticle, will then be specifically targeted to tissues having elevated expression of the thiamine receptor. Such target cells may include cancer cells.

In some preferred embodiments, the targeting agent is a protein associated to the particle via the linker. When referring to immunonanoparticles, the targeting agent is preferably an antibody associated with the particle via covalent binding to the linker (the linker being non-covalently attached to the particle). The other member of the binding couple is an antigen to which the antibody specifically binds. As indicated above, the targeting agent may also be an immunological fragment of an antibody.

In the context of the present invention, the term “antibody” means a substantially intact immunoglobulin derived from natural sources, from recombinant sources or by the use of synthetic means as known in the art, all resulting in an antibody which is capable of binding an antigenic determinant. The antibodies may exist in a variety of forms, including, e.g., polyclonal antibodies, monoclonal antibodies, single chain antibodies, light chain antibodies, heavy chain antibodies, bispecific antibodies or humanized antibodies; as well as immunological fragments of any of the above [Antibodies: A Laboratory Manual, Cold Spring Harbor Laboratory Press, NY; Harlow et al. (1989), Antibodies: A Laboratory Manual, Cold Spring Harbor, N.Y.; Houston et al. (1988), Proc. Natl. Acad. Sci. USA 85: 5879-5883; Bird et al. (1988), Science 242: 423-426)].

As used herein, the term “immunological fragment” refers to a functional fragment of an antibody that is capable of binding an antigenic determinant. Suitable immunological fragments may be, for example, a complementarity-determining region (CDR) of an immunoglobulin light chain (“light chain”), a CDR of an immunoglobulin heavy chain (“heavy chain”), a variable region of a light chain, a variable region of a heavy chain, a light chain, a heavy chain, an Fd fragment, and immunological fragments comprising essentially whole variable regions of both light and heavy chains, such as Fv, single-chain Fv (scFv), Fab, Fab′, F(ab)₂ and F(ab′)₂.

According to a preferred embodiment of the invention, the antibody is a monoclonal antibody (MAb). The antibody may be a native protein or a genetically engineered product (i.e. recombinant antibody) or an antibody produced against a synthetic product. Preferably, the ligand is a humanized and/or chimeric monoclonal antibody or a humanized and/or chimeric monoclonal antibody fragment.

According to the invention, the ligand is in particular an antibody or an antibody fragment directed against an antigen selected from the group consisting of EpCAM, VEGF, EGFR, HER2, HER3, HER4, CA 125, CTLA-4, H-Ferritin, wherein the ligand is preferably selected from the group consisting of Trastuzumab, Cetuximab, Bevacizumab, Panitumumab, Matuzumab, Nimotuzumab, MDX-447, Oegovomab, Pertuzumab, Ipilimumab, AMB8LK, anti-mouse EpCAM, anti-human EpCAM.

Non-limiting examples of MAb which may be used in accordance with the invention are Bevacizumab, Omalizumab, Rituximab, Trastuzumab (all Genentech Inc.), monoclonal antibody 17-1A (EpCAM) (BD), anti-CD160 and AMB8LK (MAT Evry, France), Muromonab-CD3 (Johnson&Johnson), Abciximab (Centocor), Rituximab (Biogen-IDEC), Basiliximab (Novartis), Infliximab (centrocor), Cetuximab (Imclone Systems), Daclizumab (Protein Design Labs), Palivizumab (MedImmune), Alemtuzumab (Millenium/INEX), Gemtuzumab ozogamicin (Wyeth), Ibritumomab tiuxetan (Biogen-IDEC), Tositumomab-1131 (Corixa) and Adalimumab (Abbot).

More preferably the MAb is EpCAM. EpCAM is a MAb with high affinity towards the epithelial cell adhesion molecule, the latter over-expressed in malignant cells, such as in lung cancer cells. Thus, according to one embodiment of the invention, the delivery system may be used to delivery a cytotoxic agent to cells over expressing cell adhesion molecule.

According to some embodiments, the NP's carry two antibodies with different binding properties (e.g. different binding specificities). This structure of two different antibodies on a single nanoparticle created a “functional bispecific-like” antibody construct where the two antibodies are placed in vicinity to each other by the nanoparticle, in a relatively simple and inexpensive manner, without the need to chemically conjugate or genetically engineered a truly bi-specific single molecule

In this context, also diabodies may be used. Diabodies are a class of small bivalent and bispecific antibody fragments that can be expressed in bacteria (E. coli) and yeast (Pichia pastoris) in functional form and with high yields. Diabodies comprise a heavy (VH) chain variable domain connected to a light chain variable domain (VL) on the same polypeptide chain (VH-VL) connected by a peptide linker that is too short to allow pairing between the two domains on the same chain. This forces paring with the complementary domains of another chain and promotes the assembly of a dimeric molecule with two functional antigen binding sites. To construct bispecific diabodies the V-domains of antibody A and antibody B are fused to create the two chains VHA-VLB, VHB-VLA. Each chain is inactive in binding to antigen, but recreates the functional antigen binding sites of antibodies A and B on pairing with the other chain.

The nanoparticles of the present invention can be formed by various methods, for example: polymer interfacial deposition method, solvent evaporation, spray drying, coacervation, interfacial polymerization, and other methods well known to those ordinary skilled in the art.

Preferably the nanoparticles of the present invention are prepared by polymer interfacial deposition method as described by Fessi H et al. [Fessi H. et al. Int. J. Pharm. 1989; 55: R1-R4, The nanoparticles of the present invention may be prepared as disclosed in U.S. Pat. Nos. 5,049,322 and 5,118,528].

According to the procedure by Fessi H. et al. the particle forming polymer is dissolved in a water-miscible organic solvent: such as acetone, tetrahydrofuran (THF), acetonitrile. To this polymer containing organic phase a linker as defined above is added. The resulting organic phase is then added to an aqueous phase containing a surfactant to form dispersion, following by mixing at 900 rpm, for 1 hour, and then evaporated under reduced pressure to form nanoparticles which are then washed with a suitable buffer, such as phosphate buffered saline (PBS). The organic phase may also comprise other surfactants as well as a combination of organic solvents so as to facilitate the dissolution of an active agent to be carried by the delivery system of the invention. Similarly, the aqueous phase may contain a combination of surfactants, all of which being as described by Fessi et al.

As indicated, the delivery particle preferably carries one or more active agents, wherein the one or more active agent(s) is/are preferably a drug and/or a radiopharmaceutical and/or a contrasting agent. To this end, the agent(s), preferably dry active agent, is added to the organic phase prior to, or together with, the addition of the linker.

In order to enable formation of the nanoparticles the polymer and active agent (if incorporated) should preferably be soluble in the organic phase and insoluble in an aqueous phase, while the organic solvent and aqueous phase should be miscible.

It was found that by mere mixing the above three components, i.e. the particle forming polymer, the active agent and the linker, an amount the linker is exposed at the surface of the particle, which amount is sufficient to allow chemical binding of a targeting agent at the surface of the particles. Thus, to the forming particles (loaded with an active agent) a targeting agent is chemically associated by providing suitable conditions to allow its cross-reaction with the reactive group of the linker, exposed at the surface of the particle.

FIGS. 1A-1C are schematic illustrations of a delivery particle according to some embodiments of the invention. FIG. 1A provides a delivery particle (10) having at its outer surface (12) a linker (14) having a first portion (16) anchored in the particle through the outer surface, and a second portion (18) exposed at said surface, to which a targeting agent (20) is chemically bound. In this particular illustration, the linker is OMCCA, having a lipophilic anchored in the particle, and a maleimide moiety exposed at the surface. Maleimide may be chemically bound to the targeting agent via the formation of e.g. a sulfide bridge with a free thiol group at the targeting agent. FIG. 1B illustrates a delivery particle identical to that of FIG. 1A, however, having at its surface hydrophilic groups (22), such as PEG, to, inter alia, increase the circulation time of the particle in the body as appreciated by those versed in the art of drug delivery vehicles. FIG. 1C illustrates a delivery particle identical to that of FIG. 1B, however also indicating that a drug (24) is embedded within the internal matrix (26) of the particle.

It will be appreciated that while FIGS. 1A-1C illustrate that the first portion of the linker is fully embedded in the particle, this portion may also be partially entrapped in the particles' matrix or entrapped or encapsulated in the core. The only prerequisite is that the anchoring is essentially stable, i.e. that the linker cannot desorb from the particle.

There is a wide variety of active agents which may be carried by the delivery particle of the invention. Carrying may be achieved by conjugation or embedment of the active agent (cluster or non-clusters of the active agent) in the polymer matrix, adsorption at the surface of the particle, dispersion of the active agent in the internal space of the particle, dissolution of the active agent within the polymer forming the particle, encapsulation in the oily core of the nanoparticle etc., as known to those versed in the art.

The active agent may be a drug (therapeutic or prophylactic agent), or a diagnostic (contrasting) agent. Active agents to be administered in an aerosol formulation are preferably selected from the group consisting of proteins, peptide, bronchodilators, corticosteroids, elastase inhibitors, analgesics, anti-fungals, cystic-fibrosis therapies, asthma therapies, emphysema therapies, respiratory distress syndrome therapies, chronic bronchitis therapies, chronic obstructive pulmonary disease therapies, organ-transplant rejection therapies, therapies for tuberculosis and other infections of the lung, fungal infection therapies, respiratory illness therapies associated with acquired immune deficiency syndrome, an oncology drug, an anti-emetic, an analgesic, and a cardiovascular agent.

According to the invention, the drug is preferably a chemotherapeutic agent, in particular an antineoplastic chemotherapy drug or a chemopreventive drug.

According to the invention it is preferred, in particular in the therapy of lung cancer or bronchial dysplasia, if the drug is selected from the group consisting of Paclitaxel, Gefitinib, Erlotinib, Etoposide, Carboplatin, Docetaxel, Vinorelbine tartrate, Cisplatin, Doxorubicin, Ifosfamide, Vincristine sul fate, Gemcitabine hydrochloride, Lomustine (CCNU), Cyclophosphamide, Methotrexate, Topotecan hydrochlorid, irinotecan, 5-fluorouracil, Zileuton, Celecoxib, and their derivatives, wherein the derivatives of said drugs are preferably fatty acid derivatives, in particular palmitic acid derivatives, such as Paclitaxel palmitate may be.

Within the context of lung cancer according to the invention it is particularly preferred, if the drug is selected from the group consisting of Paclitaxel, Etoposide, Carboplatin, Docetaxel, Vinorelbine tartrate, Cisplatin, Doxorubicin, Ifosfamide, Vincristine sul fate, Gemcitabine hydrochloride, Lomustine (CCNU), Cyclophosphamide, Methotrexate, Topotecan hydrochlorid, the derivatives of said drugs, and combinations of said drugs or their derivatives.

Within the context of dysplasia according to the invention it is preferred if the drug is selected from the group of the chemopreventive drugs Zileuton, Celecoxib, and their derivatives.

The term “dysplasia” according to the invention is directed to low grade and/or high grade dysplasia, wherein “low grade dysplasia” is particularly directed to a lesion having minimal aberration inside the cell, and “high grade dysplasia” also comprises mild or medium dysplasia. The term “bronchial dysplasia” according to the invention is in particular directed to lung dysplasia.

The term “lung” or “lungs” according to the invention is in particular directed to the respiration organs of mammals, in particular of mice and preferably of human beings. More particular, the term “lung” concerns the respiration organ of a mouse or preferably of a human being who is in need of a therapy or diagnosis of lung cancer or bronchial dysplasia, such as a murine or preferably human patient suffering from or being susceptible to non small cell lung cancer.

In another preferred embodiment of the invention, in particular in the diagnosis and/or therapy of lung cancer or bronchial dysplasia, the active agent is a radiopharmaceutical selected from the group consisting of Calcium-47, Carbon-11, Carbon-14, Chromium-51, Cobalt-57, Cobalt-58, Erbium-169, Fluorine-18, Gallium-67, Gallium-68, Hydrogen-3, Indium-111, Iodine-123, Iodine-131, Iron-59, Krypton-81m, Nitrogen-13, Oxygen-15, Phosphorus-32, Samarium-153, Selenium-75, Sodium-22, Sodium-24, Strontium-89, Technetium-99m, Thallium-201, Xenon-133, Yttrium-90, and substances comprising at least one of said radionuclides.

For the use in diagnosis or imaging methods, particularly by PET and/or CT, it is preferred if the radiopharmaceutical is Technetium-99m (e.g. in Technetium-99m scintigraphy or CT) or Fluorine 18-FDG (e.g. in Fluorine 18-FDG PET), according to the invention.

In a further preferred embodiment of the invention, in particular in the diagnosis of lung cancer or bronchial dysplasia, the active agent is a contrasting agent selected from the group consisting of iodine-, gadolinium-, magnetite-, or fluorine-containing contrasting agents, wherein the contrasting agent is preferably selected from the group of the iodine-containing agents, in particular from the group consisting of iopromide, ioxitalamate, ioxaglate, iohexyl, iopamidol, iotralon, and metrizamide.

Anti-cancer active agents are preferably selected from alkylating agents, antimetabolites, natural products, hormones and antagonists, and miscellaneous agents, such as radiosensitizers.

Examples of alkylating agents include: (1) alkylating agents having the bis-(2 chloroethyl)-amine group such as, for example, chlormethine, chlorambucile, melphalan, uramustine, mannomustine, extramustinephoshate, mechlore-thaminoxide, cyclophosphamide, if osfamide, and trifosfamide; (2) alkylating agents having a substituted aziridine group such as, for example, tretamine, thiotepa, triaziquone, and mitomycin; (3) alkylating agents of the alkyl sulfonate type, such as, for example, busulfan, piposulfan, and piposulfam; (4) alkylating N-alkyl-N-nitrosourea derivatives, such as, for example, carmustine, lomustine, semustine, or; streptozotocine; and (5) alkylating agents of the mitobronitole, dacarbazine and procarbazine type.

Examples of anti-metabolites include: (1) folic acid analogs, such as, for example, methotrexate; (2) pyrimidine analogs such as, for example, fluorouracil, floxuridine, tegafur, cytarabine, idoxuridine, and flucytosine; and (3) purine derivatives such as, for example, mercaptopurine, thioguanine, azathioprine, tiamiprine, vidarabine, pentostatin, and puromycine.

Examples of natural products include: (1) vinca alkaloids, such as, for example, vinblastine and vincristine; (2) epipodophylotoxins, such as, for example, etoposide and teniposide; (3) antibiotics, such as, for example, adriamycine, daunomycine, doctinomycin, daunorubicin, doxorubicin, mithramycin, bleomycin, and mitomycin; (4) enzymes, such as, for example, L-asparaginase; (5) biological response modifiers, such as, for example, alpha-interferon; (6) camptothecin; (7) taxol; and (8) retinoids, such as retinoic acid.

Examples of hormones and antagonists include: (1) adrenocorticosteroids, such as, for example, prednisone; (2) progestins, such as, for example, hydroxyprogesterone caproate, medroxyprogesterone acetate, and megestrol acetate; (3) estrogens, such as, for example, diethylstilbestrol and ethinyl estradiol; (4) anti-estrogens, such as, for example, tamoxifen; (5) androgens, such as, for example, testosterone propionate and fluoxymesterone; (6) anti-androgens, such as, for example, flutamide; and (7) gonadotropin-releasing hormone analogs, such as, for example, leuprolide.

Examples of miscellaneous agents include: (1) radiosensitizers, such as, for example, 1,2,4-benzotriazin-3-amine 1,4-dioxide (SR 4889) and 1,2,4-benzotriazine 7-amine 1,4-dioxide (WIN 59075); (2) platinum coordination complexes such as cisplatin and carboplatin; (3) anthracenediones, such as, for example, mitoxantrone; (4) substituted ureas, such as, for example, hydroxyurea; and (5) adrenocortical suppressants, such as, for example, mitotane and aminoglutethimide.

In addition, the anticancer agent can be an immunosuppressive drug, such as, for example, cyclosporine, azathioprine, sulfasalazine, methoxsalen, and thalidomide. Analgesic active agents, include, for example, an NSAID or a COX-2 inhibitor. Exemplary NSAIDS that can be formulated in particle of the invention include, but are not limited to, suitable nonacidic and acidic compounds. Suitable nonacidic compounds include, for example, nabumetone, tiaramide, proquazone, bufoxamac, flumizole, epirazole, tinoridine, timegadine, and dapsone. Suitable acidic compounds include, for example, carboxylic acids and enolic acids. Suitable carboxylic acid NSAIDs include, for example: (1) salicylic acids and esters thereof, such as aspirin, diflunisal, benorylate, and fosfosal; (2) acetic acids, such as phenylacetic acids, including diclofenac, alclofenac, and fenclofenac; (3) carbo- and heterocyclic acetic acids such as etodolac, indomethacin, sulindac, tolmetin, fentiazac, and tilomisole; (4) propionic acids, such as carprofen, fenbulen, flurbiprofen, ketoprofen, oxaprozin, suprofen, tiaprofenic acid, ibuprofen, naproxen, fenoprofen, indoprofen, and pirprofen; and (5) fenamic acids, such as flutenamic, mefenamic, meclofenamic, and niflumic. Suitable enolic acid NSA/Ds include, for example: (1) pyrazolones such as oxyphenbutazone, phenylbutazone, apazone, and feprazone; and (2) oxicams such as piroxicam, sudoxicam, isoxicam, and tenoxicam.

Exemplary COX-2 inhibitors include, but are not limited to, celecoxib (SC-58635, CELEBREX, Pharmacia/Searle & Co.), rofecoxib (MK 966, L-74873 1, VIOXX, Merck & Co.), meloxicam (MOBIC@, co-marketed by Abbott Laboratories, Chicago, Ill., and Boehringer Ingelheim Pharmaceuticals), valdecoxib (BEXTRA@, G.D. Searle & Co.), parecoxib (G.D. Searle & Co.), etoricoxib (MK-663; Merck), SC-236 (chemical name of 4-&lsqb; 5-(4-chlorophenyl)-3-; (trifluoromethyl)-1H-pyrazol-1-yl)&rsqb; benzenesulfonamide; G.D. Searle & Co., Skokie, Ill.); NS-398 (N-(2-cyclohexyloxy-4-nitrophenyl)methane sulfonamide; Taisho Pharmaceutical Co., Ltd., Japan); SC-58125 (methyl sulfone spiro(2.4)hept-5-ene I; i Pharmacia/Searle & Co.); SC-57666 (Pharmacia/Searle & Co.); SC-558 (Pharmacia/Searle & Co.); SC-560 (Pharmacia/Searle & Co.); etodolac (Lodine, Wyeth-Ayerst Laboratories, Inc.); DFU (5,5-dimethyl-3-(3-fluorophenyl)-4-(4-i methylsulfonyl)phenyl 2(5H)-furanone); monteleukast (MK-476), L-745337 ((5 methanesulphonamide-6-(2,4-difluorothio-phenyl)-1-indanone), L-761066, L-761000, L-748780 (all Merck & Co.); DUP-697 (5-Bromo-2-(4-fluorophenyl)-3-(4 (methylsulfonyl)phenyl; DuPont Merck Pharmaceutical Co.); PGV 20229 (1-(7-tertbutyl-2,3-dihydro-3,3-dimethylbenzo(b)furan-5-yl)-4-cyclopropylbutan-1-one; Procter; & Gamble Pharmaceuticals); iguratimod (T-614; 3-formylamino-7-&rsqb; methylsulfonylamino-6-phenoxy-4H-1-benzopyran-4-one; Toyama Corp., Japan); BF 389 (Biofor, USA); CL 1004 (PD 136095), PD 136005, PD 142893, PD 138387, and PD 145065 (all Parke-Davis/Warner-Lambert Co.); flurbiprofen (ANSAID; Pharmacia & Upjohn); nabumetone (FELAFEN; SmithKline Beecham, plc); flosulide (CGP 28238; Novartis/Ciba Geigy); piroxicam (FELDANE; Pfizer3; diclofenac (VOLTAREN and CATAFLAM, Novartis); lumiracoxib (COX-189; Novartis); D 1367 (Celltech Chiroscience, plc); R 807 (3 benzoyldifluoromethane sulfonanilide, diflumidone); JTE-522 (Japan Tobacco, Japan); FK-3311 (4′-Acetyl-2′ (2,4-difluorophenoxy) methanesulfonanilide), FK 867, FR 140423, end FR 115068 (all Fujisawa, Japan); GR 253035 (Glaxo Wellcome); RWJ 63556 (Johnson & Johnson); RWJ 20485 (Johnson & Johnson); ZK 38997 (Schering); S 2474 ((E)-(5)-(3,5-di-tert butyl-4-hydroxybenzylidene)-2-ethyl-1,2-isothiazolidine-1,1-dioxide indomethacin; I Shionogi & Co., Ltd., Japan); zomepirac analogs, such as RS 57067 and RS 104897 (Hoffmann La Roche); RS 104894 (Hoffmann La Roche); SC 41930 (Monsanto); pranlukast (SB 205312, Ono-1078, ONON, ULTAIR@; SmithKline Beecham); SB 209670 (SmithKline Beecham); and APHS (heptinylsulfide).

A description of these classes of drugs and diagnostic agents and a listing of species within each class can be found, for instance, in Martindale, The Extra Pharmacopoeia, Twenty-ninth Edition (The Pharmaceutical Press, London, 1989), which is incorporated herein by reference in its entirety. The drugs or diagnostic agents are commercially available and/or can be prepared by techniques known in the art.

Poorly water soluble drugs which may be suitably used in the practice of the subject invention include but are not limited to beclomethasone, budesonide, ciprofloxacin, cisplatin, clarithromycin, etoposide, fluconazole, itraconazole, ketoconazole, ketoprofen, methylprednisolone, mometasone, nabumetone, norfloxacin, paclitaxel, piroxicam, triamcinolone, docetaxel, or pharmaceutically acceptable salts of any of the above-mentioned drugs.

Diagnostic agents can also be delivered by the delivery particle of the invention. Diagnostic agents may be administered alone or combination with one or more drugs as described above. The diagnostic agent can be labeled by various techniques. The diagnostic agent may be a radiolabelled compound, fluorescently labeled compound, enzymatically labeled compound and/or include magnetic compound or other materials that can be detected using techniques such as X-ray, ultrasound, magnetic resonance imaging (MRI), computed tomography (CT), or fluoroscopy.

According to one preferred embodiment the active agent to be delivered by the delivery system of the invention is a cytotoxic drug (anti-tumor agents). Cytotoxic agents exemplified herein are docetaxel, paclitaxel and paclitaxel palmitate. Specific cytotoxic agent is docetaxel (DCTX), which is known to be one of the preferred drug of choice for treating non small cell lung cancer (NSCLC).

It is appreciated that in some cases the delivery particle may comprise more than one active agent. Further, the particle may be loaded with an active agent and a suitable adjuvant therefore, i.e. an ingredient that facilitates or modified the action of the principle active agent. For example, in immunotherapy, the adjuvant will be a substance included in a vaccine formulation to enhance or modify the immune-stimulating properties of a vaccine. According to another example, the particle may comprise a combination of a drug with a multi-drug resistant (MDR) inhibitor agent to potentiate the drug action; such combination may include Verapamil known to inhibit MDR to e.g. cyclosporine A (CsA).

Further, it may occur that the targeting agent has also a therapeutic or diagnostic benefit. Thus, according to some embodiments, the particle may include only the targeting agent as the principle active agent, or in addition to the targeting agent an active agent embedded in the particle's matrix or core. Examples where the targeting agent may serves also as the active principle is trastuzumab, which is also specifically exemplified hereinbelow.

The immononanoparticles of the present invention are advantageous since they are capable of selectively binding to specific receptors or antigens and release the active agent at the desired site. The binding of the targeting agent to specific receptors or antigens triggers the transfer of the nanoparticles across biological barriers using endogenous receptor mediated transcytosis and endocytosis systems. This will improve the therapeutic efficacy of the immunoparticles preparation when absent of the targeting agent as well as reduce adverse side effects associated with the active agent.

Nanoparticles undergo rapid clearance following IV administration by the reticuloendothelial system (RES). In order to inhibit the uptake of the nanoparticles by the RES, the nanoparticles may be modified at their surface with a hydrophilic polymer. The attachment of the hydrophilic polymer to the polymer forming the particle may be a covalent or non-covalent attachment, however, is preferably via the formation of a covalent bond to a linker anchored in the surface of the particle. The linker may be the same or different from the linker to which the targeting agent is bound. The outermost surface coating of hydrophilic polymer chains is effective to provide a particle with a long blood circulation lifetime in vivo.

According to one embodiment, the hydrophilic polymer is bound to a lipid, thus forming a lipopolymer, where the lipid portion anchors in the particle's surface. Within the context of the invention, in particular for use in the therapy or diagnosis of lung cancer, a delivery system is preferred, wherein
the nanoparticle is based on pegylated poly(lactide acid);
the linker is octadecyl-4-(maleimideomethyl)cyclohexane-carboxylic to which a monoclonal anti EP-CAM antibody, in particular anti human or anti mouse EP-CAM antibody, has been coupled; and/or
the drug is palmitate Paclitaxel,
   wherein a combination of all of said features is particularly preferred for the therapy of lung cancer.

The delivery system of the invention may be utilized for therapy or diagnosis, i.e. for targeted delivery of an active principle to a target site (cell or tissue). Thus, the invention also provides a pharmaceutical composition comprising the delivery system of the invention. According to one embodiment, the pharmaceutical composition is for the treatment or prevention of a disease or disorder, the delivery system being combined with physiologically and a pharmaceutically acceptable carrier.

The term “treatment or prevention” as used herein denotes the administering of a an amount of the active agent within the delivery system effective to ameliorate undesired symptoms associated with a disease, to prevent the manifestation of such symptoms before they occur, to slow down the progression of the disease, slow down the deterioration of symptoms, to enhance the onset of remission period of a disease, slow down the irreversible damage caused in a progressive chronic stage of a disease, to delay the onset of said progressive stage, to lessen the severity or cure a disease, to improve survival rate or more rapid recovery, or to prevent a disease form occurring or a combination of two or more of the above.

The term “effective amount” in accordance with this embodiment is an amount of the active agent embedded in the delivery particle in a given therapeutic regimen which is sufficient to treat a disease or disorder. For example, when treating cancer, the amount of the active agent, e.g. cytotoxic drug, is an amount of drug loaded delivery particles which will result, for example, in the arrest of growth of the primary tumor, in a decrease in the rate of occurrence of metastatic tumors, or a decrease in the number of metastatic tumors appearing in the individual or in a decrease in the rate of cancer related mortality. Alternatively, when the drug loaded delivery system is administered for cancer prevention, an effective amount will be an amount of said particles which is sufficient to inhibit or reduce the occurrence of primary tumors in the treated individual. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art. For example, the amount may depend on the type, age, sex, height and weight of the patient to be treated, the condition to be treated, progression or remission of the condition, route of administration and the type of active agent being delivered.

The effective amount is typically determined in appropriately designed clinical trials (dose range studies) and the person versed in the art will know how to properly conduct such trials in order to determine the effective amount. As generally known, an effective amount depends on a variety of factors including the mode of administration, type of polymer and other components forming the nanoparticle, the reactivity of the active agent, the type and affinity of the targeting agent to its corresponding binding member, the delivery systems' distribution profile within the body, a variety of pharmacological parameters such as half life of the active agent in the body after being released from the nanoparticle, on undesired side effects, if any, on factors such as age and gender of the treated subject, etc.

In this case, for treatment purposes the drug loaded delivery particles of the invention may be administered over an extended period of time in a single daily dose (e.g. to produce a cumulative effective amount), in several doses a day, as a single dose for several days, etc. so as to prevent the damage to the nervous system.

In a particular preferred embodiment, the delivery system according to the invention is used as an inhalant, for the lung delivery, in particular for in vivo pulmonary administration.

The term “inhalant” according to the invention is in particular directed to all forms of aerosols, dispersions, or colloids, of the delivery system according to the invention, which are breathed in and absorbed through the lungs.

The term “aerosol” according to the invention is particularly directed to a suspension of the delivery system according to the invention in a gas or to a suspension of liquid droplets containing the delivery system according to the invention in a gas. Within this context, the term “aerosol” according to the invention may also refer to an aerosol spray. The term “aerosol spray” within the context of the invention in particular concerns any type of dispensing system which creates an aerosol mist of liquid particles containing the delivery system according to the invention, wherein this is used with a can or bottle (container) that contains a liquid under pressure. Thus, the term “aerosol” according to the invention may also relate to an aerosol spray can (or bottle) and the output of such a can (or bottle), containing the delivery system according to the invention.

In particular, the use of the delivery system as an aerosol has unexpectedly shown a particular efficient local lung delivery. Thus, the delivery system as an aerosol for local lung delivery is particularly preferred according to the invention.

In a preferred embodiment of the invention, the delivery system is used in methods for treatment of the human or animal body by therapy or in diagnostic methods practised on the human or animal body.

Accordingly, the delivery system is provided as a therapeutic or diagnostic agent, being preferably formed as an inhalant, more preferably as an aerosol, for local lung delivery.

The therapy within the context of the invention is preferably an aerosol therapy, in particular by pulmonary administration, more preferably by inhalation, of an aerosol containing the delivery system according to the invention. Thus, the pulmonary administration according to the invention is preferably an aerosol administration.

Respectively, the diagnosis within the context of the invention is preferably based on pulmonary administration, more preferably on inhalation, of an aerosol containing the delivery system according to the invention.

In a particular preferred embodiment, the delivery system according to the invention is used in the therapy or diagnosis of cancer and/or dysplasia, preferably of lung cancer and/or bronchial dysplasia, in particular of Non Small Cell Lung Cancer.

Accordingly, the delivery system is provided as a therapeutic or diagnostic agent, being preferably formed as an inhalant, more preferably as an aerosol, for the therapy or diagnosis of cancer and/or dysplasia, preferably of lung cancer and/or bronchial dysplasia, in particular of Non Small Cell Lung Cancer, by local lung delivery.

In another preferred embodiment the delivery system according to the invention is used in an imaging method, preferably in PET, such as by examining the glucose metabolism in tumor-PET, wherein FDG-PET is particularly preferred, or in CT, in the therapy or diagnosis of cancer and/or dysplasia, preferably of lung cancer and/or bronchial dysplasia, in particular of Non Small Cell Lung Cancer.

Accordingly, the delivery system is provided as a diagnostic agent for an imaging method (in particular for PET, scintigraphy or CT), being preferably formed as an inhalant, more preferably as an aerosol, for the diagnosis of cancer and/or dysplasia, preferably of lung cancer and/or dysplasia, in particular of Non Small Cell Lung Cancer, by local lung delivery.

In a further preferred embodiment, the delivery system according to the invention, preferably comprising a drug, as described herein, is used for the production of a medicament, in particular for the therapy of cancer and/or dysplasia, preferably of lung cancer and/or bronchial dysplasia, in particular of Non Small Cell Lung Cancer, wherein the medicament is preferably formed as an inhalant, in particular as an aerosol.

In another preferred embodiment, the delivery system according to the invention, preferably comprising a radiopharmaceutical, as described herein, and/or a contrasting agent, as described herein, is used for the production of a diagnostic agent, in particular for the diagnosis of cancer and/or dysplasia, preferably of lung cancer and/or bronchial dysplasia, in particular of Non Small Cell Lung Cancer, wherein the diagnostic agent is preferably formed as an inhalant, in particular as an aerosol.

As indicated above, the nanoparticles according to the present invention may be administered in conjunction with one or more pharmaceutically acceptable carriers. The properties and choice of carrier will be determined in part by the particular active agent, the particular nanoparticle, as well as by the particular method used to administer the composition. Preferably the route of administration of the delivery system of the invention is inhalation.

The nanoparticles can be inhaled in a solid state or dispersed in a physiologically acceptable diluent in a pharmaceutical carrier, such as a sterile liquid or mixture of liquids, including water, saline, aqueous dextrose and related sugar solutions, glycerol with or without the addition of a pharmaceutically acceptable surfactant, and other pharmaceutical adjuvants.

A person skilled in the art would readily be able to determine the appropriate concentrations of the active agent, amounts and route of administration to deliver an efficacious dosage of the active agent over time. Furthermore, one skilled in the art may determine treatment regimens and appropriate dosage using the nanoparticles of the present invention, inter alia, depending upon the level of control over release of the entrapped or encapsulated active agent.

Considering the above, the invention also provides a method for treating a disease related to the lung or disorder comprising administering to a subject in need an effective amount of the drug-loaded delivery system of the invention.

The types of conditions which may be treated with the delivery system of the invention are numerous, as appreciated by those versed in the art. A non-limiting list of conditions include cancer, conditions associated with the inflammatory states (inflammation or auto-immune conditions) such as infections, allergic states (e.g. bronchial asthma, drug hypersensitivity); respiratory diseases (symptomatic sarcoidosis, loeffler's syndrome, aspiration pneumonitis, tuberculosis).

Another aspect of the invention concerns a method of producing the delivery system according to the invention, comprising
preparing the polymer-based nanoparticle by the solvent displacement method, preferably by using the polymer mPEG-PLA MW 100,000 Da;
adding the linker, preferably OMCCA, to the polymer prior to the nanoparticle formation;
coupling the active agent, preferably a monoclonal anti-human EpCAM or anti-mouse EpCAM antibody, to the linker subsequent to the nanoparticle formation;
bringing the polymer and/or the nanoparticle into contact with the drug;
and, preferably, producing an aerosol or aerosol spray containing the delivery system, in particular comprising filling a liquid which contains the delivery system of the invention into a container (can or bottle) and putting said liquid under pressure, and, in particular, a valve of the container is opened, the liquid is forced out of a small hole and emerges as an aerosol or mist containing the delivery system according to the invention.

In accordance with one embodiment, the invention provides a method for the treatment of cancer, by targeting, by appropriate MAbs the delivery system loaded with an anti-cancer drug (e.g. irinotecan, docetaxel and paclitaxel palmitate) to target cells in the lung.

The present invention additionally relates to a method of imaging in a subject's body a target cell or target tissue in the lung, the method comprising:

(a) providing said subject with a delivery system of the invention carrying a contrasting agent, wherein the immunonanoparticles are associated with one or more targeting agents effective to target said delivery system to said target cell or target tissue;
(b) imaging said contrasting agent in said body.

As indicated above, the delivery system of the invention may comprise a combination of a contrasting agent (imaging agent) and a therapeutic agent. Thus, by the use of the targeting system of the invention, a dual effect may be achieved, whereby the delivery of a drug may also be imaged.

The delivery device of the invention loaded with a contrasting agent may be utilized in different imaging techniques typically employed in medical diagnostics. Such include, without being limited thereto, X-ray (computer tomography (CT) of CAT scan), ultrasound, γ-scintigraphy or MRI imaging.

The contrasting agent may be any agent known in the art of imaging. An example includes, without being limited thereto, coumarin-6, fluorescent polymer conjugates such as FITC, rhodamine, gadolinium derivates.

As appreciated, while the invention is described in this detailed description with reference to pharmaceutical and diagnostic compositions, it is to be understood that also encompassed within the present invention is the use of the delivery system for other applications and in other forms.

As used in the specification and claims, the forms “a”, “an” and “the” include singular as well as plural references unless the context clearly dictates otherwise. For example, the term “an antibody” includes one or more different antibodies and the term “a contrasting agent” includes one or more contrasting agents.

Further, as used herein, the term “comprising” is intended to mean that the delivery system include the recited elements, but not excluding others. The term “consisting essentially of” is used to define the delivery system that include the recited elements but exclude other elements that may have an essential significance on the treatment or imaging procedure. “Consisting of” shall thus mean excluding more than trace elements of other elements. Embodiments defined by each of these transition terms are within the scope of this invention.

Further, all numerical values, e.g. when referring the amounts or ranges of the elements constituting the device's layers, are approximations which are varied (+) or (−) by up to 20%, at times by up to 10% of from the stated values. It is to be understood, even if not always explicitly stated that all numerical designations are preceded by the term “about”.

### EXAMPLES

### Example 1

### Cross-Linker (OMCCA) Synthesis

For the synthesis of Octadecyl-4-(maleimidomethyl)cyclohexane-carboxylic amide (OMCCA), 100 mg of Sulfosuccinimidyl-4-(N-maleimidomethyl)cyclohexane-1-carboxylate (SMCC Pierce, Ill., USA) and 80 mg of stearylamine (SA, Sigma Chemical, MO, USA) were dissolved in 8 ml chloroform and in 41 ul of triethylamine (Reidel-de-Haen, Sigma-Aldrich Chemie GmbH, Steinheim, Germany and the reaction was incubated at 50° C. for 4 hours. The solution was washed three times with 1% HCl and the chloroform was evaporated under reduced pressure. The product was desiccated overnight and weighted. The yield was about 90% and linker formation was confirmed by H-NMR (Mercury VX 300, Varian, Inc., CA, USA), IR (Vector 22, Bruker Optics Inc, MA, USA) and LC-MS (Finnigan LCQDuo, ThermoQuest, NY, USA).

### H-NMR, IR and LC-MS Analysis

¹H-NMR (of OMCCA in CDCl₃): Peaks at: 0.008, 0.849, 0.0893, 1.009, 1.245, 1.450, 1.577, 2.157, 2.160, 2.167, 2.173, 2.178, 2.181, 3.349, 3.372, 6.692, 7.257 ppm

IR: Peaks at: 626.89, 695.63, 722.35, 834.46, 899.52, 910.59, 934.79, 1045.94, 1120.05, 1163.30, 1214.60, 1260.82, 1362.15, 1408.40, 1431.38, 1468.04, 1541.02, 1629.86, 1701.35, 2850.80, 2923.84, 3087.43, 3318.81, 3453.91 cm⁻¹

LC-MS: Peak at: 490.17, 491.26

The analysis of the NMR and IR spectrum confirms the formation of the linker OMCCA, while the LC-MS spectra clearly corroborates the molecular weight of the product which is 490 g/mol.

### Example 2

### Polymers Syntheses

### (A) PEG-PLA Synthesis and Characterization

PEG-PLA (5:20) was synthesized according to well known procedure as described by Bazile D. et al. [Bazile D, et al. J Pharm Sci, 84: 493-498 (1995)]. In brief, 2 g of methoxy polyethylene glycol mw 5000 (Sigma-Aldrich Chemie GmbH, Steinheim, Germany) were mixed with 12 g of D, L—lactide (Purasorb, Purac, Gorinchem The Netherlands) for 2 hours under dried conditions at 135° C.

The polymer was analyzed by H-NMR (Mercury VX 300, Varian, Inc., CA, USA) and by differential scanning calorimetry (STARe, Mettler Toledo, Ohio, USA).

Diblock polyethylene glycol (mw 5000) and polylactide (mw 20000) polymer (PEG-PLA 5:20) was synthesized as described above. Gel permeation chromatography (GPC) exhibited mw of 20000 and polydispersity index [PD.I] of 1.47. The polymer was analyzed by H-NMR and by differential scanning calorimetry (DSC).

### H-NMR and DSC Analysis

¹H-NMR (of PEG-PLA (5:20)): Peaks at: −0.010, −0.008, −0.001, 1.206, 1.543, 1.560, 1.567, 1.581, 1.591, 3.641, 5.136, 5.145, 5.159, 5.169, 5.182, 5.192, 5.207, 5.215, 5.231, 7.256 DSC (PEG-PLA (5:20) 3.98 mg):

Peak1: integral −118.88 mJ, onset 28.70° C., peak 43.24° C., heating rate 10° C./min
Peak2: integral −1234.12 mJ, onset 237.54° C., peak 273.98° C., heating rate 10° C./min
The analysis of the NMR and DSC spectrum clearly show the formation of the diblock polymer. It can be deduced that PEG is attached covalently to PLA.

### (B) Polylactide and Poly(Ethylene Glycol-Co-Lactide) Synthesis

The polymers: polylactide (PLA) and poly(ethylene glycol-co-lactide) (mPEG-PLA) were synthesized using the ring opening polymerization method in the presence of stannous 1-ethylhexanoate as catalyst (4). In case of synthesis of PLA; D,L-lactide (30 g) and benzyl alcohol (32 mg) as co-catalyst, are dissolved in 250 ml of dried toluene while in the case of synthesis of mPEG-PLA; 1.5 g of methoxy polyethylene glycol (mPEG, MW 5000) was used as co-catalyst and added to 250 ml of dried toluene containing already 30 g of D,L-lactide. The refluxing mixture was stirred over a Dean-Stark apparatus over a period of 4 h for azeotropic removal of water. Stannous 1-ethylhexanoate (245 mg) was added following the removal of the remaining water. Then, the mixture was heated to 135° C. for 4 h. The crude polymers were dissolved in methylene chloride and precipitated twice into 4 liters of cold propyl ether/petroleum ether mixture (3:2). Prior to characterization the polymers were vacuum dried. The synthesis of the co-polymer is depicted in the following Scheme 2:

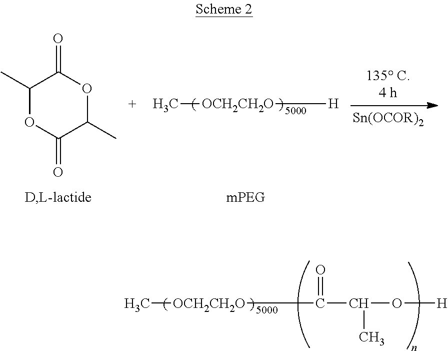

### Polylactide and Polyethylene Glycol-Co-Lactide) Characterization

The co-polymers were characterized by gel permeation chromatography (GPC) system consisting of a Waters 1515 Isocratic high performance liquid chromatography (HPLC) pump, with 2410 refractive index detector (Waters, Milford, Mass.) and a Rheodyne (Cotati, Calif.) injection valve with a 20 μl loop. Samples were eluted with chloroform through a linear Styrogel HR column, (Waters, Mass.), at a flow rate of 1 mL/min. The molecular weights were determined relative to polystyrene standards (Polyscience, Warrington, Pa.) with a molecular weight range of 54-277.7 KDa using BREEZE 3.20 version (copyright 2000, Waters Corporation computer program). Thermal analysis was determined on a Mettler TA 4000-DSC differential scanning calorimeter (Mettler-Toledo, Schwerzzenbach, Switzerland), calibrated with Zn and In standards, at a heating rate of 20° C./min under nitrogen atmosphere. ¹H-NMR spectra (in CDCl₃) were recorded on Varian 300 MHz spectrometers using TMS as internal standard (Varian Inc., Palo Alto, Calif., USA).

Polymers with molecular weights in the range of 20 000-146 000 were obtained. The basic chemical structure of PLA and mPEG-PLA polymers was confirmed by ¹H-NMR spectra which fit their composition. Overlapping doublets at 1.55 ppm are attributed to the methyl groups of the D- and L-lactic acid repeat units. The multiplets at 5.2 ppm correspond to the lactic acids CH group. When mPEG-PLA spectra is analyzed a peak at 3.65 ppm was detected which fits the methylene groups of the mPEG. According to the data obtained from the thermographs (see Table 1), only the PEG:PLA₂₀₀₀₀ exhibited crystalline domains with the appearance of a melting point thermal event at 43.2° C.

The observed crystalline domains are probably associated with the marked presence of the crystalline PEG₂₀₀₀₀ in the mPEG-PLA₂₀₀₀₀ co-polymer chain as suggested by the lack of melting point event in the thermographs of PLA₄₀₀₀₀, mPEG-PLA₁₀₀₀₀₀ and PLA₁₀₀₀₀₀ which show only a glass transition temperature, T_{g} (see Table 1). Indeed T_{g} increases with increase of PLA chains from 40000 to 100000 as noted in Table 1. It is well known that mPEG chains which are highly ordered elicit a crystalline character while PLA chains are less ordered exhibiting an amorphous state. This increase in PLA chains in the mPEG-PLA on the expense of PEG will increase the amorphous character of the co-polymers and consequently T_{g} will increase.

TABLE 1
Physical properties of synthesized polymers

| | Molecular weight ^{a} | | | |
|---|---|---|---|---|
| Polymer | Mn^{c} | Mw^{c} | T_{g} (° C.)^{b} | Tₘ (° C.)^{b} |
| PEG:PLA₂₀₀₀₀ | 20000 | 29000 | — | 43.2 |
| PEG:PLA₄₀₀₀₀ | 37000 | 52000 | 34.1 | — |
| PEG:PLA₁₀₀₀₀₀ | 87000 | 136000 | −19.4; 49.1 | — |
| PLA₁₀₀₀₀₀ | 80000 | 83000 | 65.3 | — |

^{a} molecular weight determined by GPC.
^{b}glass transition temperature (T_{g}) and melting point (Tₘ) determined by DSC.
^{c}Mn is the number average of the molecular weight and Mw is the weight average of the molecular weight.

### Example 3

### (A) Nanoparticles (NPs) Preparation and Characterization

### NP's Preparation

The PLA nanoparticles were prepare by the nanoparticles-polymer interfacial deposition method as described by Fessi H et al. [Fessi H, et al. Int. J. Pharm. 55: R1-R4 (1989)]. In brief, 88 mg of the polymer PLA (polylactide, 30 KDa purchased from Boehringer Ingelheim) and 38 mg of the co-polymer PEG-PLA, 5:20 (polyethylene glycol of MW of 5000 and polylactide MW of 20,000) were dissolved in 20 ml acetone, a water-miscible organic solvent. To this organic phase 10 mg of the drug docetaxel were added. For coupling of an antibody, to the organic phase, 20 mg of the linker OMCCA were added. The resulting organic phase was then added to 50 ml of aqueous phase which contained 100 mg Solutol® HS 15 (BASF, Ludwigshafen, Germany), as a surfactant (Macrogol 15 hydroxystearate). The dispersion was mixed at 900 rpm over 1 hr and then evaporated under reduced pressure to 20 ml. the NPs were washed with Phosphate Buffered Saline (PBS) 5-6 times using vivaspin 300 KDa cut-off. Spherical polymeric, nanometric (100-500 nm) particles were spontaneously formed under these conditions.

TABLE 2
Linker (OMCCA) containing formulation

| Organic phase | Aqueous phase |
|---|---|
| PEG (MW 5000)-polylactide (MW | Solutol ® HS 15^{(a)} |
| 20,000) [PEG-PLA 5:20] 0.88% | 0.5% w/v |
| polylactide (MW 30,000) [PLA 30] | Water 50 ml |
| 0.19% | |
| Acetone 20 ml | |
| OMCCA 0.1% w/v | |

^{(a)}Solutol^{R} HS 15 (0.5% w/v): Macrogol 15 hydroxystearate was dissolved in water at a concentration of 0.5%.

### Drug Incorporation Efficacy

Drug encapsulation (incorporation) efficacy was determined using HPLC system consisting of Kontron instruments (Watford, UK) 325 pump, Kontron instruments 332 detector adjusted at 227 nm and Kontron instruments 360 autosampler. Separation was achieved by LichroCART (Merck Darmstadt, Germany) C18 (250*4 mm, Sum) column. The mobile phase was 50% acetonitrile in water at flow rate of 1 ml/min. the retention time of docetaxel was 10 minutes.

### Nanoparticle Characterization

### (1) Particle Size Analysis

Mean diameter and particle size distribution measurements were carried out utilizing an ALV Noninvasive Back Scattering High Performance Particle Sizer (ALV-NIBS HPPS, Langen, Germany) at 25° C. and using water (refractive index: 1.332; viscosity: 0.894543) as the diluent. A laser beam at 632 nm wavelength was used. The sensitivity range was 0.5 nm to Sum.

### (2) Zeta Potential Measurements

The zeta potential of the NPs/immunoNPs was measured using the Malvern zetasizer (Malvern, UK) diluted in double distilled water.

### (3) Morphological Evaluation Using TEM

Morphological evaluation for the immunoNPs was performed by means of transmission electron microscopy (TEM) using gold labeled goat anti-human IgG (Jackson ImmunoResearch Laboratories, PA, USA).

Blank trastuzumab immunoNPs (containing no active ingredient) were incubated with a gold labeled anti-human IgG and negatively stained with phosphotungstic acid (PTA) 2% pH 6.4.

### Results

### Drug Incorporation Efficiency

The encapsulation efficiency of the cytotoxic drug docetaxel (DCTX) in the nanoparticles and in the immunonanopartricles was determined by HPLC and found to be 100% and 49%, respectively prior to purification. It was interesting to note that the theoretical drug content of the DCTX loaded NPs, 7.4% w/w (initial weight ratio PLA: PEG-PLA: DCTX; 88:38:10) was significantly higher than the drug content of DCTX immunonanoparticles, 3.3%, w/w (initial weight ratio PLA: PEG-PLA: DCTX: OMCCA; 88:38:10:20). This marked difference in DCTX content may be attributed to the presence of the linker in the polymeric matrix. During nanoparticle formation, the linker probably competes with DCTX and reduce its incorporation extent from 7.4 to 3.3%.

### Particle Size Analysis

The average and particle size distribution of the various NPs was measured using the ALV method. It was observed that the mean diameter of the blank NPs (containing no active ingredient) was 60 nm while the diameter was 150 and 180 nm for the blank immunoNPs (containing no active ingredient) and for DCTX loaded immunoNPs, respectively. The marked increase in diameter of the NPs should be related to the linker's presence which probably decreases the acetone diffusion towards the aqueous phase allowing the formation of larger NPs.

### Zeta Potential Measurements

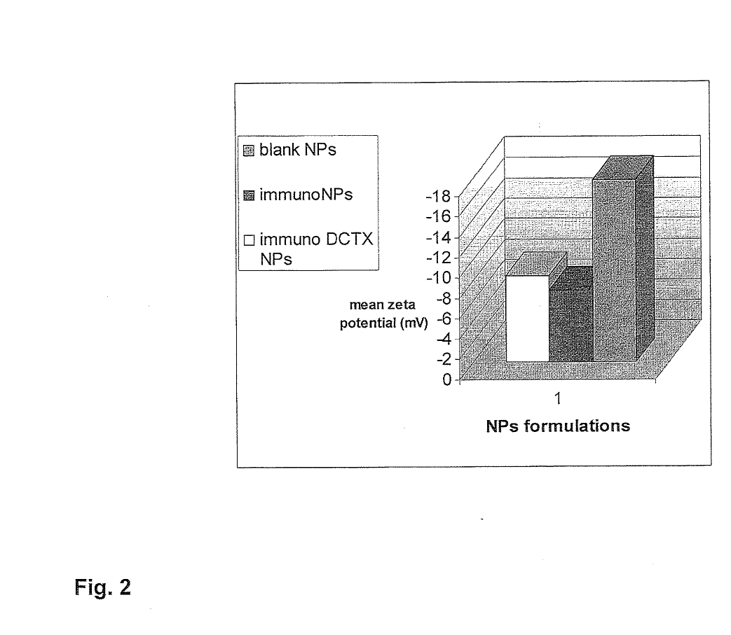

The zeta potential of the blank NPs was −18 mV and decreased to −7 mV for the antibody conjugates NPs (FIG. 2). The decrease in zeta potential should be attributed to the positive charge of trastuzumab at pH 7.4 since its isoelectric point is 9.

### Morphological Evaluation Using TEM

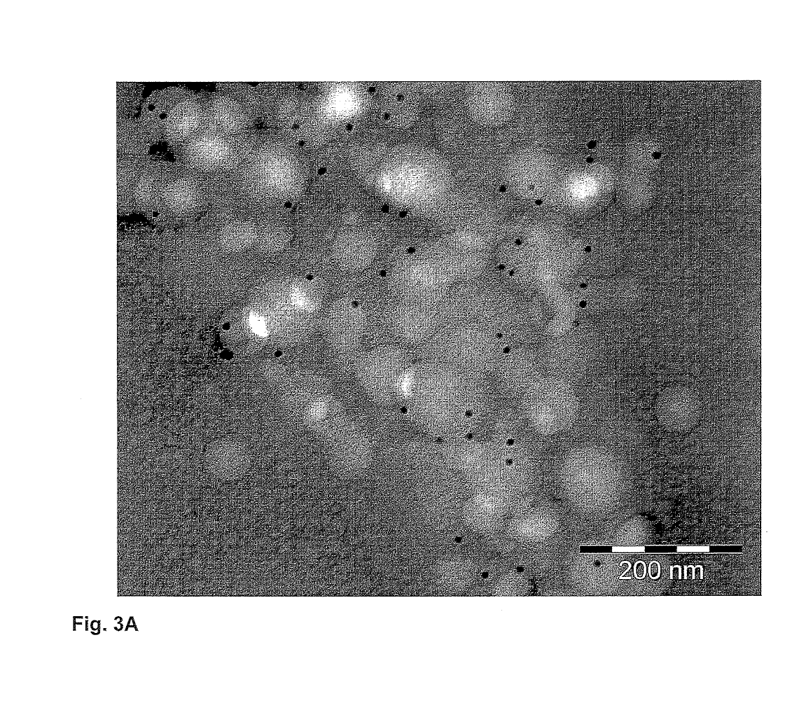

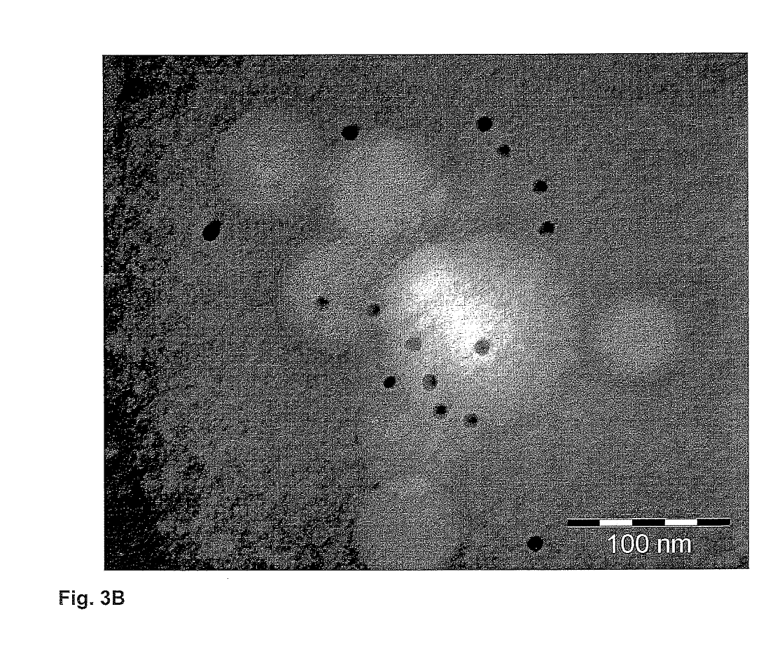

It can be noted from the results depicted in FIGS. 3A-3B that each gold black spot represents one trastuzumab molecule attached to the nanoparticle surface. It can be deduced that the MAb has been efficiently conjugated to the surface of the nanoparticle by the linker and the reaction conditions did not affect the initial affinity of the MAb to the secondary antibody

(B) Conjugation with Targeting Moiety

### Antibody Modification for Thiol Groups Generation

Increment of thiol groups on the MAb was preformed using the 2-iminothiolane reagent [Traut's reagent, Sigma-Aldrich Chemie GmbH, Steinheim, Germany, Traut R R, et al. Biochemistry. 12(17):3266-73 (1973); Jue R, et al. Biochemistry. 17(25):5399-406 (1978)]. Traut reagent was incubated for 45 min with purified trastuzumab at molar ratio of 30:1, respectively. The Traut modified MAb was separated on HiTrap desalting column (Amersham Bioscience, Uppsala, Sweden). Fractions containing the modified MAb were determined by UV at 280 nm. Free thiol groups were determined with 5,5′-dithio-bis(2-nitrobenzoic acid) (Ellman's reagent, Sigma-Aldrich Chemie GmbH, Steinheim, Germany), by monitoring the change in absorbance at 412 nm. Once reacted with Traut's reagent, mAb possess reactive sulfhydryls that can be used in conjugation protocols with sulfhydryl-reactive cross-linking reagents bearing a maleimide group such as OMCCA. The following Scheme (3) illustrates a possible conjugation reaction between reduced antibody and maleimide group of the linker:

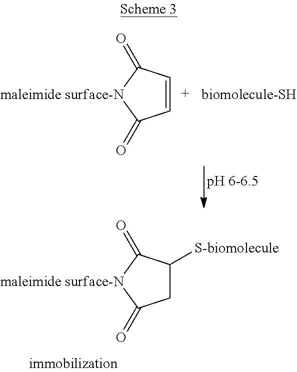

### Coupling Reaction

Freshly prepared nanoparticles consisting of 88 mg of the polymer PLA (polylactide, 30 KDm 38 mg of the co-polymer PEG-PLA, 5:20, 0 or 10 mg of the drug docetaxel and 20 mg of the cross-linker OMCCA equivalent to an overall amount of blank nanoparticles of 146 mg or 156 mg of DCTX nanoparticles (PLA: PEG-PLA: DCTX: OMCCA; 88:38:0/10:20) were adjusted to pH 6.5 with 0.1N NaOH and incubated with Traut modified trastuzumab (final concentration 1 mg/ml) overnight at 4° C. under continuous agitation and under nitrogen atmosphere. Unreacted maleimide groups were blocked through incubation with 2-mercaptoethanol (Pierce, Ill., USA) for 30 min. Unconjugated antibody and 2-mercaptoethanol were separated from immunonanoparticles by gel filtration over a Sepharose CL-4B column (Amersham Bioscience, Uppsala, Sweden). Coupling efficiency was evaluated by the BCA protein assay (Bicinchoninic Acid protein assay) (Pierce, Ill., USA) as described [Smith P. K., et al. Anal. Biochem. 150:76-85 (1985)].

For preparation of immunonanoparticles with various amounts of conjugated antibody, the initial ratio of Traut modified trastuzumab to maleimide-activated particles was varied. The actual investigated ratio was 146 mg of blank NPs or 156 mg of DCTX NPS for 26 mg of MAb.

Morphological evaluation for the final immunonanoparticles was performed by means of transmission electron microscopy (TEM) using gold labeled goat anti-human IgG (Jackson ImmunoResearch Laboratories, PA, USA).

### Drug Content Determination

The final drug content in the nanoparticles was evaluated as follows: the colloidal dispersion comprising a final volume of 20 ml is first ultrafiltrated using Vivaspin of 30000 daltons cutoff (Sartorius, Goettingen, Germany) to obtain 2-3 ml of clear ultrafiltrate. The concentration of DCTX in the ultrafiltrate is measured by HPLC. The remaining total volume of colloidal dispersion is then lyophilized, weighted and subjected to total DCTX content analysis using HPLC for final calculation of drug content in the nanoparticles. Various initial increasing drug ratios will be tested to identify the optimal formulation. Furthermore, the presence of possible tiny drug crystals in the colloidal dispersion will be also monitored.

### Absorption of Trastuzumab to Blank Nanoparticles

The purpose of this determination was to evaluate whether trastuzumab molecules are physically absorbed onto blank nanoparticles, i.e. nanoparticles containing no linker anchored at their surface. To this end, 100 ul (1 mg) of trastuzumab 7.5 mg/ml solution were mixed over 1 hour at room temperature with 1 ml of blank positive and negative charged nanoparticle aqueous dispersions containing a total amount of 125 mg nanoparticles. The mixture (750 ul of) was then washed 5 times with 30 ml of PBS and the diluted dispersion was filtered through vivaspin 300 KDa cut-off using centrifugation (4000 rpm, 30 min) to remove unabsorbed MAb molecules.

The protein concentration was determined using PCA protein assay to detect the presence of MAb molecules in the nanoparticle supernatant.

### Results

### SH Group Determination

The number of sulfhydryl groups on the modified MAb was determined using Ellman's reagent compared to cysteamine as standard. The intact trastuzumab and the Traut modified trastuzumab were diluted with PBS buffer containing 0.1M EDTA pH 8 and incubated with Ellman's reagent. The Traut modified trastuzumab SH groups per MAb was determined to be 31.5 as compared to 1.4 in the intact trastuzumab.

### Coupling Efficiency Determination

The amount of the MAb conjugated to the NPs was determined using BCA protein assay. NPs were degraded with 0.1N NaOH at 50° C. and incubated with assay reagent. The coupling efficiency for the immunoNPs (without the drug DCTX) and for the immuno DCTX loaded NPs was 71 and 77%, respectively.

### Absorption of Trastuzumab to Blank Nanoparticles

The ratio between the amount of trastuzumab before and after separation for the positive and negative formulations was 4.2 and 2.7%, respectively.

These results ensure that there was no absorption of MAb molecules onto the nanoparticles following successive washings with PBS and therefore the coupling of MAb to linker containing nanoparticles is most probably mediated by a covalent conjugation since all the successive washings and purification processes during immunonanoparticle preparation are carried out using PBS at similar dilution extent.

The lack of MAb adsorption on vivaspin membranes was validated in previous experiments when MAb aqueous solutions were subjected to identical experimental conditions and the concentrations of MAb in the supernatant and ultrafiltrate were found to be similar.

### Example 4

### (A) NP's Preparation for In Vivo Study

### 1. Nanoparticles Preparation

Nanoparticles were prepared using the polymer interfacial deposition method. The organic phase contained 300 mg of the polymer mPEG-PLA MW 100,000 dalton (Sigma, St. Louis, Mo.), 100 mg of Tween 80 (Sigma, St. Louis, Mo.) and 20 mg stearylamine (Sigma, St. Louis, Mo.) that were dissolved in 50 ml acetone (J. T. Baker, Deventer, Netherlands). The organic phase was added to 100 ml of an aqueous solution of 100 mg Solutol® HS 15 (BASF, Ludwigshafen, Germany). The suspension was stirred at 900 rpm for 1 h and subsequently concentrated by evaporation to 10 ml. All formulations were diafiltrated with a 100-ml solution of 0.1% Tween 80 (Vivaspin 300,000 MWCO, Vivascience, Stonehouse, UK) and filtered through a 1.2-um filter (FP 30/1.2 CA, Schleicher & Schuell, Dassel, Germany). A typical cationic NP formulation consisted (in % w/w) of mPEGPLA 100,000, Solutol® HS 15 1, stearylamine 0.2, Tween 80 1 and doubled-distilled water to 100. The composition of the anionic NP formulation was identical to that of the cationic NP with the exception of the cationic lipid (i.e. lacking stearylamine).

### 2. Immunonanoparticles Preparation

For preparation of immunoNPs the cross linker OMCCA (20 mg) was added to the polymer within the organic phase prior to NPs formation. Rat anti mouse EpCAM antibody (IgG2Ak, batch G8.8 11/2004) was originally dissolved in Tris azide buffer in a concentration of 10 mg/ml. Salts removal and dissolving in Borate buffer pH=8-8.5 was performed using nanosep centrifugal device 30K (Ann Arbor, Mich., USA). The antibody was then was mixed with 2-iminothiolane, also known as Traut's reagent (Pierce, Rockford, Ill.) at a molar ratio of 1:50 in Borate buffer (pH 8) for 1 h at 4° C. The solution was purified from excess Traut's reagent by gel filtration using Sephadex G-25 HiTrap desalting column (Amersham Bioscience, Uppsala, Sweden). Antibodies were collected in 0.3 ml fractions. Fractions containing mAb were determined using UV at 280 nm, pooled together and kept under nitrogen atmosphere at 4° C. until coupling to anionic NPs.

Freshly prepared anionic NPs were adjusted to pH 8 and incubated with Traut modified EpCAM antibody (final antibody concentration 1 mg/ml) overnight at 4° C. in a nitrogen environment under mild shaking. The formulation was then diafiltrated with 60 ml solution of 0.1% Tween 80 (Vivaspin 300,000 MWCO, Vivascience, Stonehouse, UK). Coupling efficiency was evaluated by the BCA protein assay. All formulations prepared were stored at 4° C. in sterile glass bottles under nitrogen atmosphere. Isotonicity of the tested NP formulations was adjusted using 5% glucose.

Nanoparticles characterization by size and zeta potential determination was performed as described above.

Results

The zeta potential and size values of different formulations are presented in Table 3. The uniformity in size of the different NPs formulations evaluated, ranging from 129 nm to 148 nm, is important since previous studies revealed surface charge dependent as well as size and surface area effects on pulmonary nanoparticulates toxicity (Tetley, 2007).

ImmunoNPs were manufactured by conjugation of EpCAM MAb to PEG-PLA NPs based on experience gained during the conjugation of MAbs to cationic emulsions and NPs in Prof. Benita's group. The total amount of EpCAM mAb conjugated to NPs was evaluated with the BCA protein assay kit. EpCam antibody conjugation efficiency was 77% (based on BCA in NaOH) and 80% (based on direct method BCA in DDW).

TABLE 3
Physico-chemical properties of NPs
and immunoNPs.

| | | EpCAM |
|---|---|---|
| Parameter | NPs | immunoNPs |
| Mean diameter, | 129.3 | 148.3 |
| nm | | |
| Mean zeta | −30.7 | −27.6 |
| potential, mV | | |

### (B) Safety Evaluation of Pulmonary Delivered immunoNPs in Murine Model

Non invasive local pulmonary delivery was performed by means of endotracheal instillation using a mouse model. Five consecutive daily endotracheal administrations into mice lungs were performed and safety evaluation was carried out at 8 (3 days recovery) and 19 days (14 days recovery) from the first administration. During 5 days of instillation period, mice were weighed daily

The procedures involving healthy female BALB/c mice (8-10 weeks old) were approved by The Authority for Animals Facilities of The Hebrew University of Jerusalem based on guidelines from the NIH guide for the Care and Use of Laboratory Animals were followed for all experiments.

Administration was conducted via the endotracheal route using MicroSprayer™ aerosoliser (IA-1C; Penn-Century, Philadelphia, Pa., USA) suitable for mice, attached to a high-pressure syringe (FMJ-250; Penn-Century) as described before (Bivas-Benita et al., 2005). Prior to the procedure, mice were anaesthetized by an intraperitoneal injection of ketamine and xylazine (Fort Dodge, Iowa, USA). The method was validated following visual examination of extracted mice thorax as reported by Bivas-Benita et al.

The dose range concentration was selected based on Dailey et al. study which investigated the pro-inflammatory potential of diethylaminopropylamine polyvinyl alcohol-grafted polylactic-co-glycolic acid) (DEAPA-PVAL-g-PLGA) NPs instilled (and not endotracheally applied) in mice lungs

### Results

### 1. Method Validation:

All the mice evaluated (i.e. 10/10) for dye localization within the mice thorax immediately after endotracheal application exhibited specific lungs staining without any extra-pulmonary staining.

### 2. Mice Survival and Weight Change

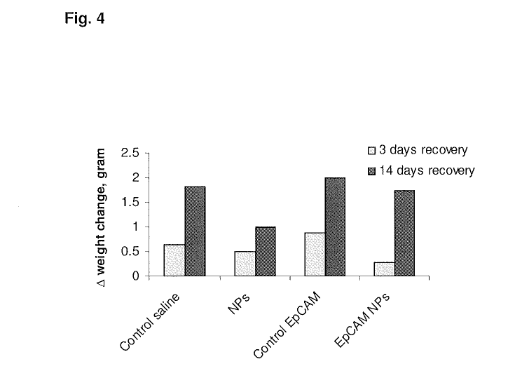

During the 5 days of NPs application and the following 3 or 14 days recovery non of the animals died as a result from the NP application. Weight reduction or non-gaining weight is a general marker for possible toxicity. Fluctuations in weight during 5 days of instillation were observed although were not statistically significant. Non significant weight changes among the different treatments were also depicted following 3 and 14 days recovery as presented in FIG. 4.

FIG. 4: Mice weight change following 3 and 14 days recovery in mice instilled with NPs over 5 consecutive days

### (C) Bronchoalveolar Lavage (BAL) Cell Analysis

Bronchoalveolar lavage (BAL) was performed as described before (Segel et al., 2005). Briefly, mice were scarified using an intraperitoneal injection of lethal dose of pentobarbital (Pental Veterinary; CTS Chemical Industries, Tel Aviv, Israel). BAL was centrifuged for 10 min, 1000 rpm. Total BAL cell count was performed using a hemocytometer. A differential cell count was performed on 200 cells by using cytospin slides stained with Diff-Quik (Baxter). Cells numbers for the different BAL cell types are the products of the total cell count and the percentages obtained from the differential count, expressed as cells/ml of BAL fluid and analyzed for total cell count, differential cell count and macrophage

### Results

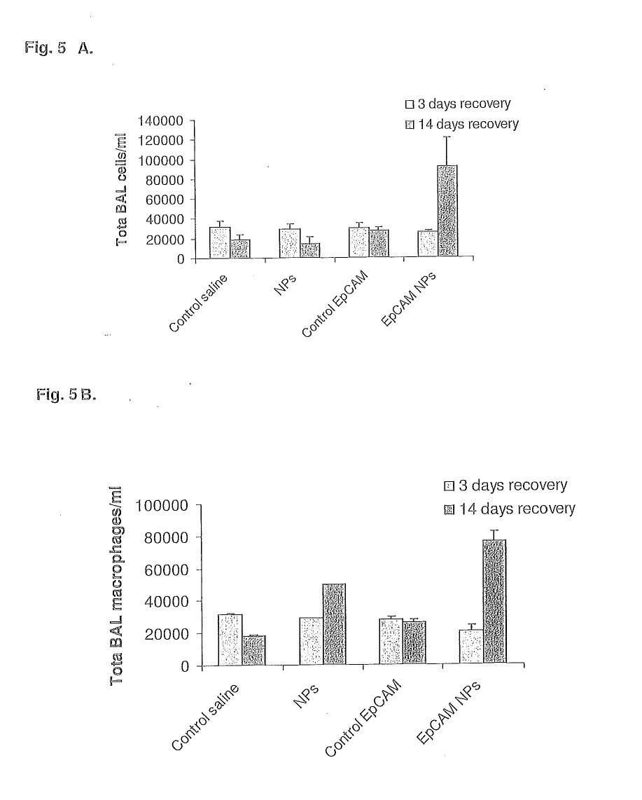

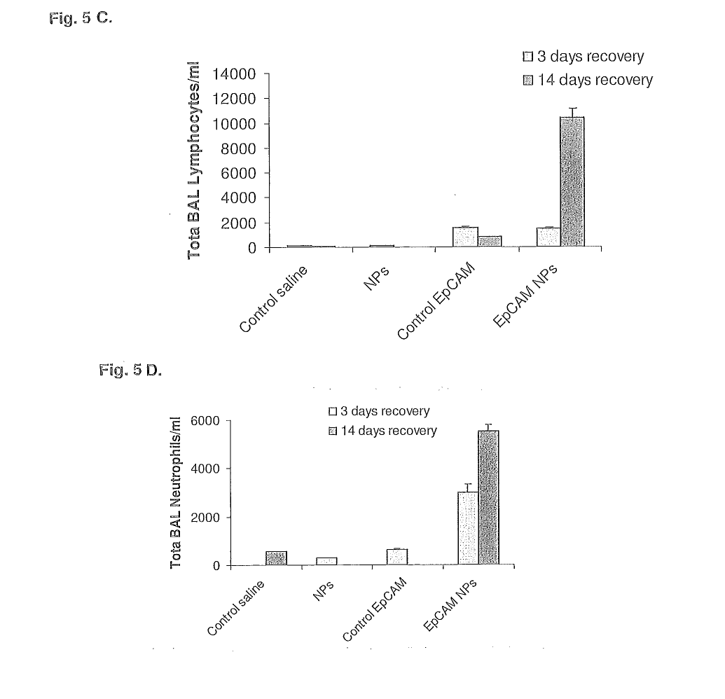

The total BAL values of PEG-PLA NPs were similar to that of the control in both 8 and 19 days experiment (FIG. 5A). Increased macrophages levels were observed at 14 days recovery. EpCam conjugated NPs application did not result in increased total BAL cells initially and cell values were similar to that of the control and the anionic PEG-PLA NPs (FIG. 5A). However, increased neutrophil and lymphocyte counts within the BAL were observed (FIG. 5B-D). Furthermore, long term total cell number in BAL increased significantly compared to the saline control or the antibody saline control and also compared to the anionic PEG-PLA NPs (FIG. 5A). Specifically, BAL macrophages, lymphocytes and neutrophils were increased (FIG. 5B-D). It appears that the effects of this formulation were noted only after 14 days recovery. It may indicate that an extended immune response took place following introduction of rat antibody to mice lungs. However, since the effects of this formulation were different from the control antibody saline, it is possible that the polymeric nature of immunoNPs conjugate may have specific effects.

FIG. 5: Mice BAL following 3 and 14 days recovery in mice instilled with NPs over 5 consecutive days. Total cell BAL (A), total BAL macrophages (B), total BAL lymphocytes (C), total BAL neutrophils (D).

### (D) Hematological Data

Following 3 and 14 days recovery blood samples were collected using a method of sub mandibular bleeding from the mice cheek pouch (Golde et al., 2005). This vein was puncture and blood was collected into K3EDTA tubes (Greiner, Kremsmunster, Austria). Complete blood count was performed at the same day using sysmex K-21 blood analyzer (Sysmex Corporation, Kobe, Japan). White blood cell differential was performed manually.

### Results

The data presented in Table 4 indicates that through 8 days of NPs application increased neutrophils and decreased lymphocyte percentage were observed, although none significantly changed compared to the other groups. The immunoNPs formulation exhibited increased stability, eosinophils and monocytes percentage as well as platelets, all within the reference value range.

Decreased platelets levels were observed during 19 days experiment for NPs (Table 5). NPs also exhibited the most elevated levels of eosinophils although levels were within the reference range. ImmunoNPs exhibited decrease in WBC levels compared to the controls, although non significant changes were observed among the various formulations evaluated. The different WBC populations exhibited specific increase in both neutrophils and monocytes accompanied with decreased lymphocytes for the immunoNPs group although variations were not statistically significant.

TABLE 4
Total Hematological parameters following 3 days recovery
in mice instilled with NPs over 5 consecutive days.

| | Control | | Control | |
|---|---|---|---|---|
| Parameter | Saline | Neg NPs | EpCAM | EpCAM NPs |
| Units | n = 6 | n = 4 | n = 7 | n = 6 |
| WBC | 4.45 ± 0.494 | 4.5 ± 0.993 | 4.343 ± 0.500 | 4.35 ± 0.640 |
| 10³ cmm | 3.2−6.1 | 2.1−6.9 | 3−6.5 | 3.3−7.5 |
| RBC | 10.34 ± 0.18 | 9.385 ± 0.428 | 10.289 ± 0.14 | 9.77 ± 0.18 |
| 10⁶ cmm | 9.94−11 | 8.14−9.67 | 9.92−11 | 9.13−10.3 |
| HGB** | 16.63 ± 0.29 | 14.58 ± 0.54 | 16.66 ± 0.09 | 15.7 ± 0.28 |
| g/dl | 16.2−17.7 | 13.1−15.6 | 16.5−17.2 | 14.9−16.8 |
| HCT | 51.93 ± 1.02 | 47.8 ± 2.52 | 51.73 ± 0.73 | 48.61 ± 1.04 |
| % | 48.5−55 | 40.7−52.1 | 49.4−55.1 | 45.4−51.6 |
| MCV** | 50.05 ± 0.34 | 50.85 ± 0.57 | 50.06 ± 0.08 | 49.67 ± 0.15 |
| fL | 49.2−51.6 | 49.8−52.2 | 49.8−50.3 | 49.2−50.2 |
| MCH | 16.03 ± 0.14 | 15.55 ± 0.43 | 16.13 ± 0.20 | 16.05 ± 0.11 |
| pg | 15.7−16.5 | 14.3−16.1 | 15−16.7 | 15.6−16.4 |
| MCHC** | 32.05 ± 0.20 | 30.6 ± 0.97 | 32.23 ± 0.41 | 32.32 ± 0.29 |
| % | 31.3−32.6 | 27.8−32.2 | 30.1−33.6 | 31.1−33 |
| PLAT* | 698 ± 39 | 582 ± 188 | 918 ± 103 | 878 ± 50 |
| 10\hat{ }³ cmm | 547−765 | 360−1143 | 767−1523 | 707−1039 |
| NEUT | 17.83 ± 5.41 | 26.25 ± 2.50 | 22.29 ± 1.85 | 21.33 ± 3.27 |
| % | 7−40 | 19−30 | 15−28 | 9−31 |
| STAB* | 0.0 ± 0.0 | 0 ± 0 | 0 ± 0 | 0.17 ± 0.17 |
| % | 0−0 | 0−0 | 0−0 | 0−1 |
| LYMP | 81 ± 5.56 | 72.75 ± 2.14 | 76.71 ± 1.77 | 76.67 ± 3.51 |
| % | 59−91 | 70−79 | 72−83 | 67−90 |
| MONO | 1 ± 0.37 | 0.75 ± 0.48 | 0.57 ± 0.30 | 1.17 ± 0.31 |
| % | 0.2 | 0 − 2 | 0 − 2 | 0 − 2 |
| EOS | 0.17 ± 0.17 | 0.25 ± 0.25 | 0.43 ± 0.43 | 0.67 ± 0.21 |
| % | 0−1 | 0−1 | 0−3 | 0−1 |
| BASO | 0.0 ± 0.0 | 0 ± 0 | 0 ± 0 | 0 ± 0 |
| % | 0−0 | 0−0 | 0−0 | 0−0 |

TABLE 5
Total Hematological parameters following 3 days recovery
in mice instilled with NPs over 5 consecutive days.

| | Control | | Control | |
|---|---|---|---|---|
| Parameter | Saline | Neg NPs | EpCAM | EpCAM NPs |
| Units | n = 5 | n = 3 | n = 8 | N = 6 |
| WBC | 4.62 ± 0.29 | 4.30 ± 0.70 | 4.59 ± 0.73 | 4.05 ± 0.35 |
| 10\hat{ }3 cmm | 3.5−5 | 2.9−5.1 | 2.1−9 | 2.9−5.1 |
| RBC** | 10.1 ± 0.14 | 10.31 ± 0.66 | 9.66 ± 0.18 | 9.83 ± 0.16 |
| 10\hat{ }6 cmm | 9.66−10.3 | 9.12−11.4 | 8.61−10.2 | 9.31−10.3 |
| HGB | 15.82 ± 0.17 | 16.6 ± 0.53 | 16.39 ± 0.33 | 15.42 ± 0.38 |
| g/dl | 15.6−16.2 | 15.6−17.4 | 14.8−17.8 | 14−16.5 |
| HCT*** | 51.92 ± 0.79 | 56.6 ± 4.41 | 48.46 ± 1.02 | 49 ± 0.79 |
| % | 49.7−53.1 | 49.7−64.8 | 43−51.9 | 46−51.1 |
| MCV*** | 51.26 ± 0.1 | 54.67 ± 1.01 | 50.1 ± 0.13 | 49.8 ± 0.18 |
| fL | 50.9−51.5 | 53−56.5 | 49.5−50.6 | 49.4−50.5 |
| MCH* | 15.62 ± 0.07 | 16.2 ± 1.10 | 16.98 ± 0.43 | 15.65 ± 0.27 |
| Pg | 15.4−15.8 | 15−18.4 | 15.9−19.6 | 15−16.3 |
| MCHC*** | 30.48 ± 0.17 | 29.63 ± 2.12 | 33.9 ± 0.8 | 31.45 ± 0.50 |
| % | 29.9−30.8 | 26.9−33.8 | 31.9−39.3 | 30.3−32.7 |
| PLAT | 837 ± 26 | 549 ± 179 | 566 ± 83 | 722 ± 60 |
| 10\hat{ }3 cmm | 764−946 | 276−888 | 349−957 | 586−974 |
| NEUT | 21.4 ± 1.86 | 19 ± 4.04 | 17.25 ± 3.58 | 28.5 ± 1.88 |
| % | 14−24 | 11−24 | 8−34 | 23−36 |
| STAB | 0 ± 0 | 0 ± 0 | 0 ± 0 | 0 ± 0 |
| % | 0−0 | 0−0 | 0−0 | 0−0 |
| LYMP | 77.6 ± 1.69 | 79 ± 3.51 | 81.75 ± 3.80 | 69.83 ± 1.83 |
| % | 74−84 | 75−86 | 65−92 | 62−75 |
| MONO | 0.6 ± 0.4 | 1.33 ± 0.7 | 0.63 ± 0.32 | 1.33 ± 0.42 |
| % | 0−2 | 0−2 | 0−2 | 0−3 |
| EOS | 0.4 ± 0.24 | 0.67 ± 0.33 | 0.38 ± 0.18 | 0.33 ± 0.21 |
| % | 0−1 | 0−1 | 0−1 | 0−1 |
| BASO | 0.0 ± 0 | 0 ± 0 | 0 ± 0 | 0 ± 0 |
| % | 0−0 | 0−0 | 0−0 | 0−0 |

### (E) Histopathology

Samples of both lungs, the heart, liver and kidney were preserved in formaldehyde. Each vial was assigned a number, with the treatment unknown to the pathologist. The lungs were fixed for at least 7 days before further processing. The formalin-fixed samples were embedded in paraffin, thin-sectioned, and mounted on glass microscope slides using standard histopathological techniques. Sections were stained with hematoxylin-eosin and examined by light microscopy.

### Results

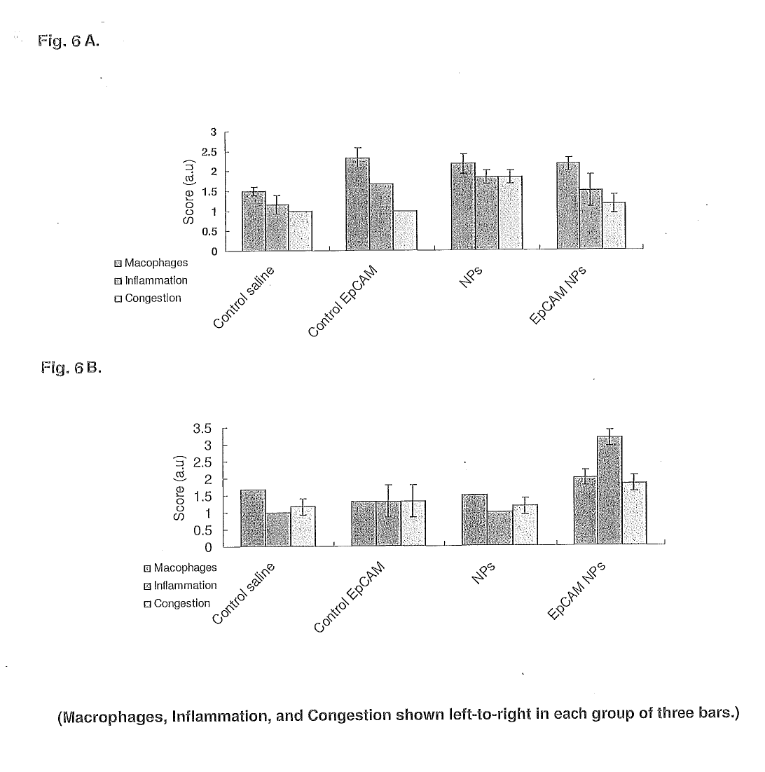

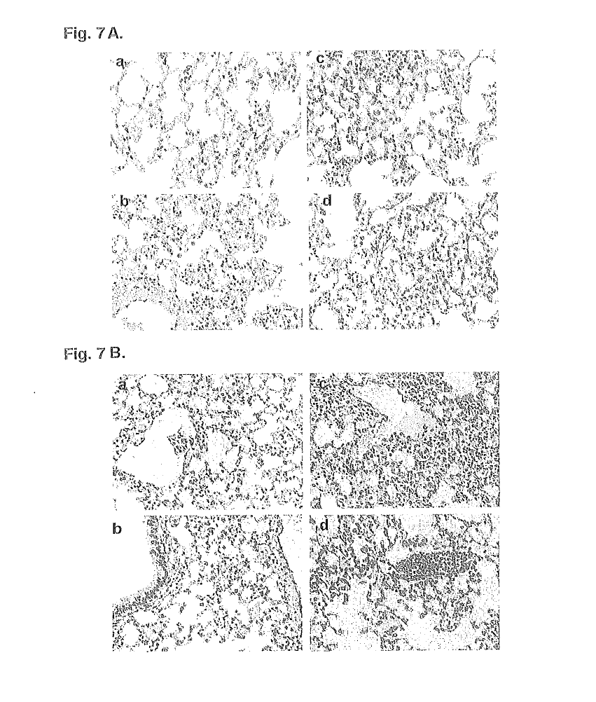

No pathologic changes were found in extrapulmonary organs i.e. heart, liver and kidney, in both short and long term experiments. Triplicates of both lungs were evaluated separately with no significant changes observed between both lungs. The pathological changes in the lung samples examined were subtle and minimal. The main finding was that of reactive macrophages in the alveolar spaces. The differences between the very small numbers of macrophages if present at all, in the various groups were barely detectable so that exact objective quantification was not practical and not possible. The same appears to the attempts of quantifying and defining the subtle differences in the very moderate findings of inflammation and congestion. However, ranking of inflammation, congestion and macrophages within lungs was performed (FIGS. 6 A and B) It can be observed that following 3 days recovery few macrophages were indeed observed in 10-20% of the alveoli within the lungs of mice applied with NPs, immunoNPs and EpCAM control and these level of macrophages remained similar only for immunoNPs during the long term experiment. Interestingly, NPs exhibited also relatively high scores of inflammation and congestion in short term experiment that were reduced to normal values through the additional 14 days of recovery. Only for immunoNPs formulation increased inflammation score was observed compared to the controls during the 19 days of experiment. Representative pictures of each group are also presented (FIGS. 7 A and B).

FIG. 6: Scoring of macrophages, congestion and inflammation within both lungs of mice installed with different formulations following 3 (A) and 14 (B) days recovery. Results presented as means±SEM, n=3. Ranking index: Macrophage: 1—few macrophages in several alveoli, 2—few macrophages in 10-20% of the alveoli, 3—grouped (2-3) macrophages in 10-20% of the alveoli, 4—grouped (2-3) macrophages in above 30% of the alveoli Inflammation: 1—no inflammation, 2—very few foci of the lymphatic aggregates (probably reactive BALT-broncho-associated lymphatic tissue), 3—few foci of chronic infiltrates dispersed in the interstitial tissue of the lung, 4—slight increase in number of foci of chronic infiltrates dispersed in the interstitial tissue of the lung. Congestion: 1—no congestion, 2—foci of moderate engorgement of capillaries in the alveolar septi, 3—foci of moderate engorgement of capillaries in the alveolar septi accompanied by slight edema and thickening of the involved septi.

FIG. 7: Lung tissues from mice instilled with different formulations and scarified on the 8 (A) and 19 (B) days of experiment. Magnifications of pictures presented are X 400. The pictures presented: a. control saline, b. PEG-PLA NPs, c. control EpCAM saline and e. EpCAM NPs.

The overall results clearly indicated that immunonanoparticles can be used for local delivery to the lung by inhalation and can elicit a therapeutic activity once used in a lung cancer diseased animal model.

Further scope of applicability and preferable features of the present invention will also become apparent from the examples given hereinafter. However, it should be understood that the detailed description and specific examples, while indicating preferred embodiments of the invention, are given by way of illustration only, since various changes and modifications within the spirit and scope of the invention will become apparent to those skilled in the art from this examples and detailed description.

### Example 5

The invention can be carried out by inhalation as well as by injection. In a preferred embodiment of the invention the nanoparticles according to the invention are administered by inhalation. The advantage of inhalation compared to intravenous application is demonstrated as follows:

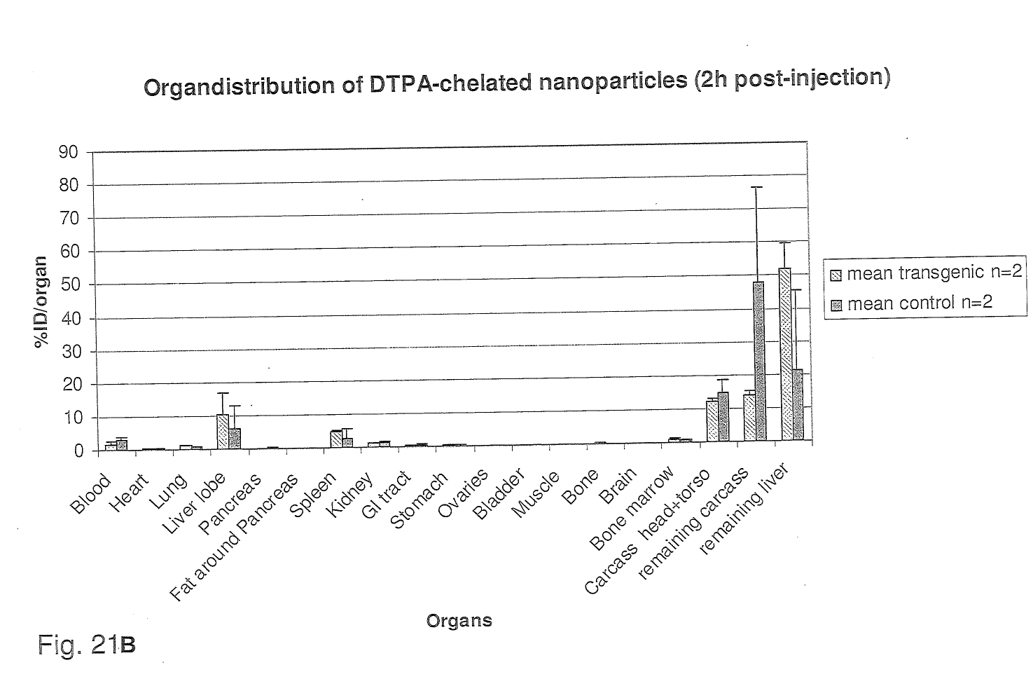

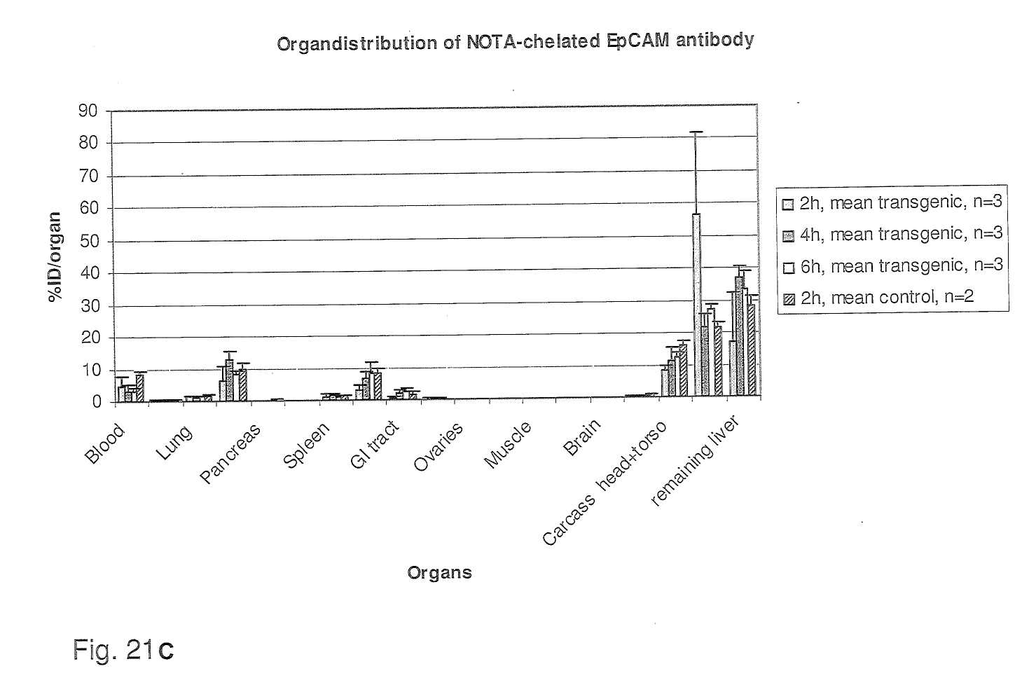

The organ distribution of 68Ga-labeled NOTA-chelated anti-EpCAM antibody as well as DOTA and DTPA-chelated nanoparticles are injected into the tail vein of either SPC/c-raf transgenic lung tumor bearing mice or non-transgenic Balb/c mice. It is shown that the active agent only marginally reaches the lungs. Most of it accumulates in the head and the torso, the carcass and the liver (see FIG. 21 a)-c)).

A huge difference between intravenous application and inhalation exists, therefore for lung delivery, inhalation is preferred.

FIG. 21 a)-c)
Depicted is the organ distribution of 68Ga-labeled NOTA-chelated anti-EpCAM antibody as well as DOTA and DTPA-chelated nanoparticles that were injected into the tail vein of either SPC/c-raf transgenic lung tumor bearing mice or non-transgenic Balb/c mice. The mice were sacrificed at different time points and the organ/tissue specific radioactivity was measured in a multiwell-counter.
A safety and tolerability study of EpCAM immunonanoparticles for local pulmonary drug delivery:

This study examined the safety and tolerability of pulmonary delivered immunonanoparticles (INP). Specifically, an EpCAM monoclonal antibody conjugated to biodegradable polymeric polyethylene glycol poly(lactic acid) (PEG-PLA) INPs was compared to plain PEG-PLA NPs. Bronchial alveolar lavages (BAL) were collected, and hematological, histochemistry and immunohistochemistry parameters were studied. This was done to assess both local and systemic effects following pulmonary administration of either EpCam conjugated PEG-PLA INPs, anionic PEG-PLA NPs or cationic PEG-PLA NPs after 5 days of daily endotracheal instillation to BALB/c mice that were sacrificed on the eighth or nineteenth day of the experiment.

Notably, the cationic PEG-PLA NPs elicited increased local and systemic toxic effects both on the eighth and nineteenth day. In contrast, anionic NPs of similar size elicited significantly lower local and systemic responses with local inflammatory effects observed only on the eighth experimental day. The EpCAM INP formulation elicited pulmonary inflammatory effects probably due to local immune response. Although the BAL results revealed recruitment of PMNs, lymphocytes and macrophages to lungs of mice applied with INPs, there were only minor local histopatholgic changes. However, immunohistochemistry suggested specific pulmonary localization of EpCAM INPs even after nineteen experimental days. Overall, these observations indicate that anionic PEG-PLA NPs exhibit potential as a pulmonary drug carrier and thus should be considered as a promising therapeutic drug delivery system for the treatment of primary and metastatic lung cancer.

Abbreviations: Bronchoalveolar lavage, BAL; D8, eighth experimental day; D19, nineteenth experimental day; Diethylaminopropylamine poly(vinyl alcohol)-grafted poly(lactic-co-glycolic acid), DEAPA-PVAL-g-PLGA; Dipalmitoylphosphatidylcholine, DPPC; Epidermal growth factor, EGF; Epithelial cell adhesion molecule, EpCAM; Food and Drug Administration, FDA; Immunonanoparticles, INPs; Monoclonal antibody, MAb; Nanoparticles, NPs; Polymorphonuclear, PMN; Severe combined immune deficiency, SCID.

Inhalation of drug loaded immunonanoparticles (INPs) may therefore prove to be extremely valuable as a result of enhanced specificity and prolonged localization in targeted lung cells

It is the objective of the present study to assess the potential adverse effects of INPs as a function of the surface charge following local pulmonary delivery by means of a non invasive endotracheal instillation as previously described by Bivas-Benita et al.²³ in a murine model. In the current study INPs were prepared using an antibody recognizing epithelial cell adhesion molecule (EpCAM). This antigen is frequently overexpressed in carcinoma tumors and specifically in carcinoma tumors of the lungs, as recently reported. To the best of our knowledge, the possible toxic effects of endotracheally delivered antibody conjugated NPs have not yet been evaluated. Therefore, possible pulmonary and systemic effects following local pulmonary delivery of differently charged and targeted PEG-PLA NPs were evaluated. Data on the safety and tolerability of INPs following inhalation are reported.

### Methods

### Nanoparticles

### A. Nanoparticle Preparation

Nanoparticles were prepared using the solvent displacement method. The organic phase contained 300 mg of the polymer mPEG-PLA MW 100,000 dalton, 100 mg of Tween 80 (Sigma, St. Louis, Mo.) and 20 mg stearylamine (Sigma, St. Louis, Mo.) that were dissolved in 50 ml acetone (J. T. Baker, Deventer, Netherlands). The organic phase was added to 100 ml of an aqueous solution of 100 mg Solutol® HS 15 (BASF, Ludwigshafen, Germany). The suspension was stirred at 900 rpm for 1 h and subsequently concentrated by vacuum evaporation to 10 ml. All formulations were diafiltrated with a 100-ml solution of 0.1% Tween 80 (Vivaspin 300,000 MWCO, Vivascience, Stonehouse, UK) and filtered through a 1.2 μm filter (FP 30/1.2 CA, Schleicher & Schuell, Dassel, Germany). A typical cationic NP formulation consisted (in w/w) of mPEG-PLA MW 100,000 3, Solutol®HS 15 1, stearylamine 0.2, Tween 80 1 and doubled-distilled water to 100. The composition of the anionic NP formulation was identical to that of the cationic NP with the exception of the cationic lipid (i.e., lacking stearylamine).

### B. Immunonanoparticles Preparation

For preparation of INPs the linker octadecyl-4-(maleimideomethyl)cyclohexane-carboxylic amide (OMCCA) (20 mg) was added to the polymer within the organic phase prior to NPs formation. Monoclonal purified rat anti-mouse CD326 antibody against EpCAM (Becton Dickinson Biosciences, Heidelberg, Germany) was originally dissolved in Tris azide buffer in a concentration of 10 mg/ml. Salts removal and dissolving in Borate buffer pH=8-8.5 was performed using a Nanosep centrifugal device 30K (Ann Arbor, Mich., USA). The antibody was mixed with 2-iminothiolane, also known as Traut's reagent (Pierce, Rockford, Ill.) at a molar ratio of 1:50 in Borate buffer (pH 8) for 1 h at 4° C. The solution was purified from excess Traut's reagent by gel filtration using Sephadex G-25 HiTrap desalting column (Amersham Bioscience, Uppsala, Sweden). The antibody was collected in 0.3 ml fractions. Fractions containing MAbs were determined using UV at 280 nm, pooled together and kept under nitrogen atmosphere at 4° C. until coupling to anionic NPs.

Freshly prepared anionic NPs were adjusted to pH 8 and incubated with Traut modified EpCAM antibody overnight at 4° C. in a nitrogen environment under mild shaking. The formulation was then diafiltrated with 100 ml solution of 0.1% Tween 80 (Vivaspin 300,000 MWCO, Vivascience, Stonehouse, UK). Coupling efficiency was evaluated by the BCA protein assay. All formulations prepared were stored at 4° C. in sterile glass bottles under nitrogen atmosphere.

### Physicochemical Characterization of Nanoparticles

Particle size distribution and mean diameter measurements were carried out using an ALV noninvasive backscattering high-performance particle sizer (ALV-NIBS HPPS, Langen, Germany) at 25° C. NPs formulations were diluted with double-distilled water as described previously.

Zeta potential measurements were carried out using a Malvern Zetasizer (Malvern Instruments, Ltd., Malvern, UK). The samples were diluted in double-distilled water as performed previously^{1,25,27}.

### Animals

Female BALB/c mice (8-10 weeks old, Harlan, Jerusalem, Israel) were used for in vivo pulmonary administration. The procedures involving animals were approved by The Authority for Animals Facilities of The Hebrew University of Jerusalem based on guidelines from the NIH for the Care and Use of Laboratory Animals. These guidelines were followed for all experiments (approval no. MD 114.20-2). Mice were housed in a specific pathogen-free environment in plastic cages on hardwood shavings, three to eight animals per cage. A 12-h light\dark cycle was maintained, and mice had access to water and rodent laboratory chow ad libitum. Mice were acclimated to these conditions for 3-7 days before treatments commenced.

### Experimental Design

The safety evaluation of 5 different formulations was performed following 5 daily endotracheal instillations into mice lungs. Two control groups: sterile saline, EpCAM antibody sterile saline solution and three NPs formulations of cationic PEG-PLA NPs, anionic PEG-PLA NPs and EpCAM-conjugated anionic PEG-PLA NPs were evaluated. Each mice group consisted of 7 animals and received the treatment over 5 consecutive days, followed by animal sacrifice and analysis at the eighth or nineteenth experiment day resulting in a total of 14 animals per formulation. The polymer dose instilled was 0.15 mg in 25 μl of 5% glucose solution.

### Endotracheal Instillation

Different nano-scaled formulations or controls either conjugated or not to an antibody were administered daily to healthy female BALB/c. Administration was conducted via the endotracheal route using MicroSprayer™ aerosoliser (IA-1C; Penn-Century, Philadelphia, Pa., USA) suitable for mice, attached to a high-pressure syringe (FMJ-250; Penn-Century, Philadelphia, Pa., USA) as previously described. Prior to the procedure, mice were anaesthetized by an intraperitoneal injection of 100 mg/kg body weight ketamine and 10 mg/kg body weight xylazine (Fort Dodge, Iowa, USA). Mice were suspended at a 45° angle by the upper teeth. The light source's (Euromex microscopes, Arnhem, Holland) flexible fiber-optics arm was adjusted to provide optimal illumination of the mouse trachea. A small spatula was used to open the lower jaw of the mouse and blunted forceps were used to help displace the tongue for maximal oropharyngeal exposure. After a clear view of the trachea was obtained, the Micro Sprayer tip was endotracheally inserted and 25 μl of solution or suspension was sprayed. The tip was immediately withdrawn and the mouse was taken off the support.

### Method Validation

Validation of endotracheal administration of formulations to the lungs was performed. Trypan blue was endotracheally applied to the lungs of 10 mice, immediately after the mice were sacrificed. Evaluation of the thorax was performed to pinpoint the blue staining. All of the mice evaluated for trypan blue localization immediately after endotracheal administration exhibited specific staining of the lungs without extra-pulmonary staining (i.e. 10 lungs from 10 mice).

### Blood Sampling and Analysis

On the eighth and nineteenth experiment day blood samples were collected using the method of sub mandibular bleeding from the mouse cheek pouch. This vein was punctured and blood was collected into K₃EDTA tubes (Greiner, Kremsmunster, Austria). A complete blood count was performed on the same day using sysmex K-21 blood analyzer (Sysmex Corporation, Kobe, Japan). White blood cell differential was performed manually. The hematological analyses were carried out by an approved and recognized sub-contractor (Herzliya Medical Center, Herzliya, Israel).

### Bronchoalveolar Lavage (BAL), BAL Cell Counting and Differential

Bronchoalveolar lavage (BAL) was performed using the technique previously described by Segel and colleagues. Briefly, mice were sacrificed using an intraperitoneal injection of a lethal dose of pentobarbital (Pental Veterinary; CTS Chemical Industries, Tel Aviv, Israel). The trachea was cannulated with a blunted 22 gauge needle and BAL was performed by means of injection and withdrawal of 4 ml sterile saline. BAL fluid was centrifuged for 10 min, 1000 rpm. Total BAL cell count was performed using a hemocytometer. A differential cell count was performed on 200 cells by using cytospin slides stained with Diff-Quik (Baxter, Mc Graw Park, Ill.).

### Histopathologic Examination

After lung lavage was completed, lungs, heart, liver and kidney were excised and preserved in formaldehyde. The lungs were fixed for at least 7 days before further processing. The fixed samples were embedded in paraffin, thin-sectioned, and mounted on glass microscope slides using standard histopathological techniques. The pathologist (Y.S.) who reviewed the slides of the lungs and other organs was blinded as to the treatment or control groups. Sections were stained with hematoxylin-eosin and examined by light microscopy in triplicates.

### Immunohistopathologic Assessment

Lung samples were dissected and fixed in 4% neutral buffered formalin. 5 μm-thick histological sections were deparaffinized, rehydrated through a graded alcohol series and washed with dH₂O for 4 minutes. After treating the sections with proteinase K (Dako, Hamburg, Germany) for 5 minutes and a 5 minute washing step in dH₂O, the endogenous peroxidase activity was blocked with peroxidase blocking reagent (Dako, Hamburg, Germany) for 5 minutes. Afterwards sections were washed with Tris-buffered saline (wash buffer) over 5 minutes and incubated with protein block serum-free reagent (Dako, Hamburg, Germany) for 10 minutes. Incubation with monoclonal purified rat anti-mouse CD326 antibody against EpCAM was performed overnight at 4° C. Following the washing step, the sections were coated with biotinylated link universal secondary antibody (DAKO LSAB+ Kit, Hamburg, Germany) for 15 minutes and washed again. Streptavidine-peroxidase-conjugate reagent (Dako-LSAB+ Kit, Hamburg, Germany) was used for visualisation of immunoreactivity. After counterstaining with hematoxylin the sections were dehydrated and coverslipped for the examination under light microscope.

### Statistics

Differences between treatments were compared using analysis of variance (ANOVA). Differences in weight gain with the same treatment among the experiment days (eighth day compared to first day and nineteenth day compared to the first day) were compared using two-tailed paired t-test.

### Results

### Physicochemical and In Vitro Characterization of Nanoparticles

Average NP diameter and zeta potential values of different formulations anionic, cationic and INPs were 129.3±1.12, 141.3±1.3, 148.3±1.8 nm and −30.7±0.9, 31.67±2.1, −27.6±0.8 mV, respectively. The conjugation efficiency of EpCAM MAb to the particles was 77% as determined by the BCA protein assay kit (Pierce Chemical, Rockford, Ill.) meaning that 176 MAb molecules were conjugated per single NP based on calculations previously reported^{30,31}. In vitro cytotoxicity assessment of positively and anionic NPs using the quantitative colorimetric MTT assay was performed both in polarized HELA, polarized epithelial MDCK and human lung adenocarcinoma A549 cells. Overall results indicated that both NP formulations, regardless of their surface charge did not elicit marked cytotoxic effects and there were no statistically significant differences in the cellular effects elicited among the differently charged formulations (data not shown). Antibody conjugated INPs compared to non targeted anionic NPs as well as EpCAM saline cytotoxicity against A549 cell line were evaluated using the MTT assay without marked effects on cells following 4 hours of exposure (data not shown).

### Mice Survival and Body Weight Gain

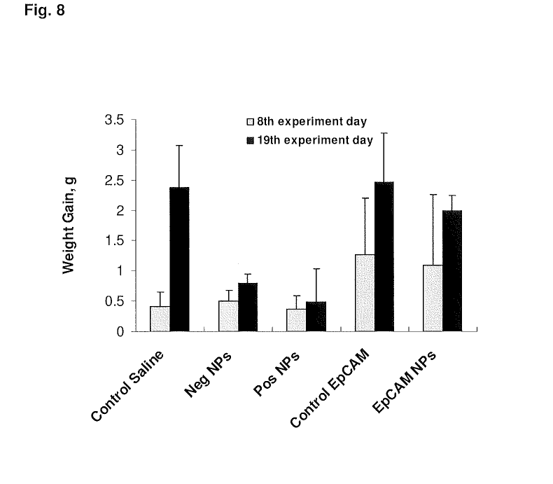

Cationic stearylamine-PEG-PLA NP was the only formulation that induced one animal death that may have been due to the increased toxicity of this formulation. No significant changes in daily measured body weight gain during 5 days of instillation of the various formulations were observed (data not shown). Inhalation of different NP formulations did not elicit significant body weight changes up to the eighth experimental day (D8) (FIG. 8). On the nineteenth experimental day (D19), the negatively and cationic NPs clearly showed decreased body weight gain. It is interesting to note that the application of anionic NP formulation resulted in reduced body weight gain on D19 as compared to the anionic INPs. Furthermore, the administration of EpCAM MAb both in the saline control solution and conjugated to NPs resulted in similar body weight gain as the control saline group (FIG. 8).

### Bronchoalveolar Lavage Assessment

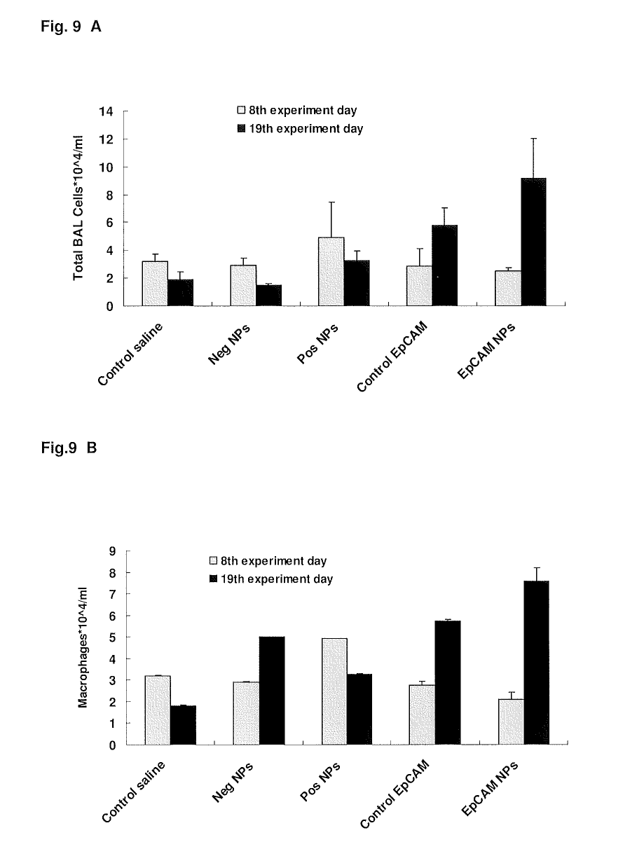

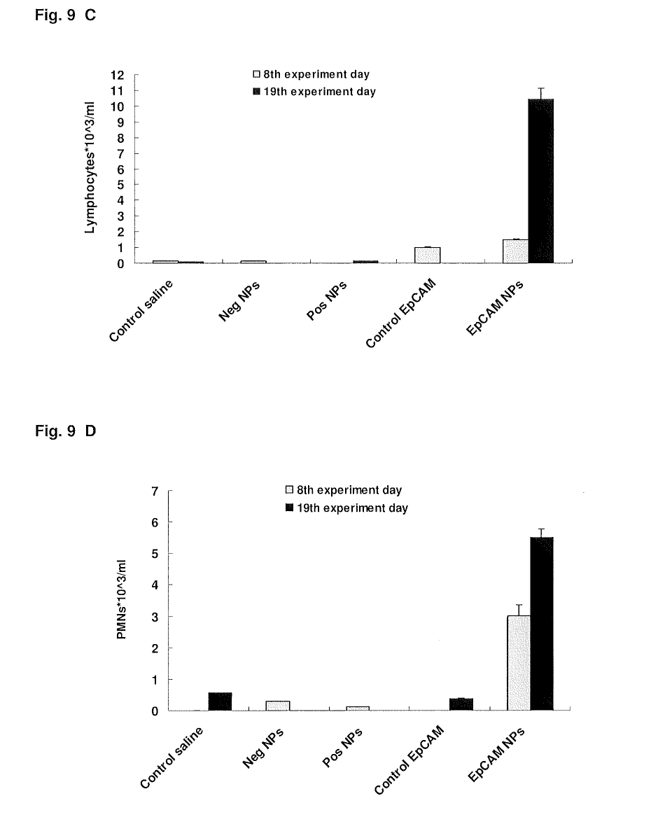

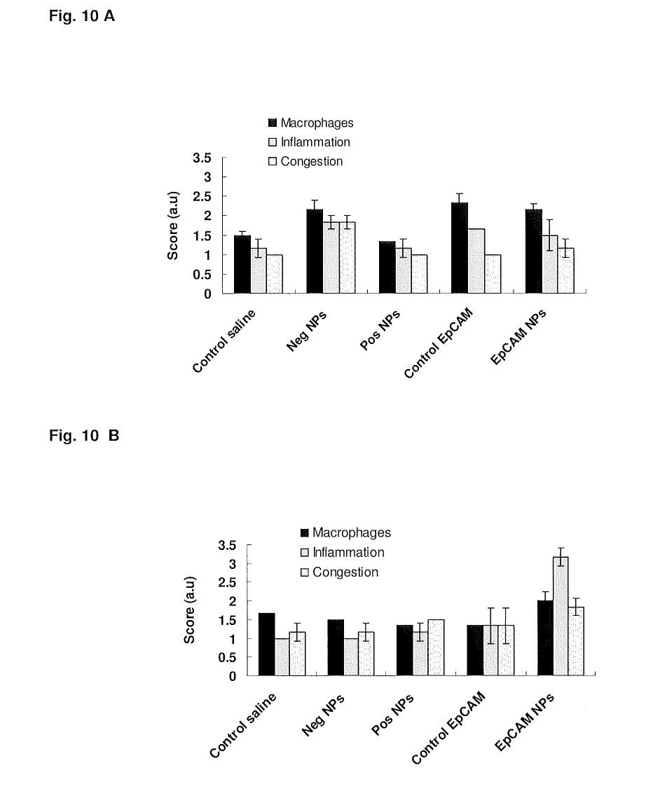

The total BAL cell count, macrophages, lymphocytes and polymorphonuclear (PMN) cells within the BAL are shown in FIG. 9 A-D, respectively. Eosinophils were not traced at all on day 0, D8 and D19. No marked differences among the various formulations in total BAL cell values were determined at D8 (FIG. 9.A). However, significant differences in macrophage, lymphocyte and PMN cell numbers (p<0.0001) were observed on D8 (FIG. 9B-D). Anionic PEG-PLA NP administration did not elicit any significant change within the various BAL cell populations as compared to the control saline. Specifically, cationic NPs in comparison to other formulations significantly elicited the BAL macrophages on D8 (p<0.0001) (FIG. 9.B). Increased PMN cells and lymphocytes together with decreased macrophage counts were observed for the INPs as compared to the control saline on D8. In addition, a marked increase in PMN cells (p<0.001) was observed with the INPs formulations as compared to the control MAb saline treatment group.

The total BAL cell count among the different formulations was significantly different (p<0.05) on D19 (FIG. 9.A). Significantly elevated macrophage counts (p<0.05) were determined for negatively as compared to control saline and cationic NPs. The INPs formulation yielded an increased cellular response within the lungs on D19. Specifically, following the application of INP as compared to all the other formulations tested (p<0.01), significantly elevated levels of macrophages, lymphocytes and PMN cells were noted on D19. These elevated counts of pulmonary macrophages, lymphocytes and PMN cells suggested a non specific local immune response within the lungs following INP administration (FIG. 9).

### Hematological Data

Overall, only moderate changes were induced in mice blood counts following the pulmonary administration of various formulations. Decreased white blood count was observed on D8 following the application of cationic NPs (Table 6). Within the white blood cell population significant differences were observed among the neutrophils (p<0.0001), stabs (p<0.0001), lymphocytes (p<0.0001), monocytes (p<0.0001) and eosinophils (p<0.0001). The moderate changes observed in platelets (p<0.05), HGB (p<0.01), MCV (p<0.01) and MCHC (p<0.01) were, in fact, significant. Specifically for cationic NPs, decreased monocyte levels were noted on D8. These findings of systemic leucopenia together with decreased systemic monocytes levels are in agreement with the findings within the lungs of increased BAL cells and macrophages specifically following the application of cationic NPs on D8. However, on D19 a 103% increase in white blood cell count was noted with cationic NPs adjusting to the normal physiological values (Table 7). However, significant differences among the different groups were noticed for neutrophils (p<0.0001), lymphocytes (p<0.0001), monocytes (p<0.0001), eosinophils (p<0.0001) and stabs declined to zero on D19. Significant differences were also observed in red blood cells values (p<0.01), HCT (p<0.001), MCV (p<0.0001), MCH (p<0.05), and MCHC (p<0.001) between the various NP formulations.

### Histological Changes in the Lung

No pathologic changes were found in extra-pulmonary organs i.e. heart, liver and kidney, both on D8 and D19. Triplicate sections of both left and right lungs were evaluated separately with no significant changes observed among them. The pathological changes in the lung samples examined were subtle and minimal. The main finding was that of reactive macrophages in the alveolar spaces. The differences between the small numbers of macrophages, inflammation and congestion if present at all, in the various groups, were so barely detectable that exact objective quantification was not practical or possible. However, semi-quantitative ranking of inflammation, congestion and macrophages within each lung was performed (FIGS. 10 A and B).

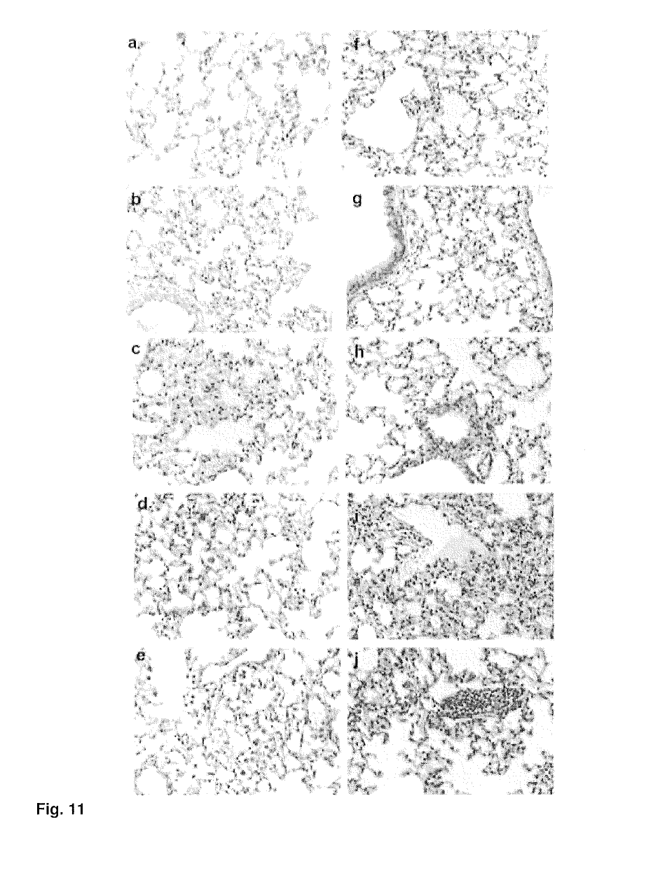

Inhalation of cationic NPs did not induce any pathologic signs within the lungs. This may be explained by increased phagocytic uptake of the cationic NPs that were not associated to the cells of the bronchial and alveolar wall. Furthermore, only few macrophages were detected in 10-20% of the alveoli within the lungs of mice applied with anionic NPs, INPs and EpCAM control saline on D8 and their number decreased by D19. In addition, anionic NPs exhibited elevated inflammation and congestion scores on D8 that were reduced to values similar to the control by D19. INPs formulation elicited an increased inflammation score compared to the controls during the nineteen days of the experiment. This is in agreement with the BAL results of this formulation (FIG. 10 B). A representative illustration of each group is presented in FIGS. 11A-J.

### Immunohistochemistry

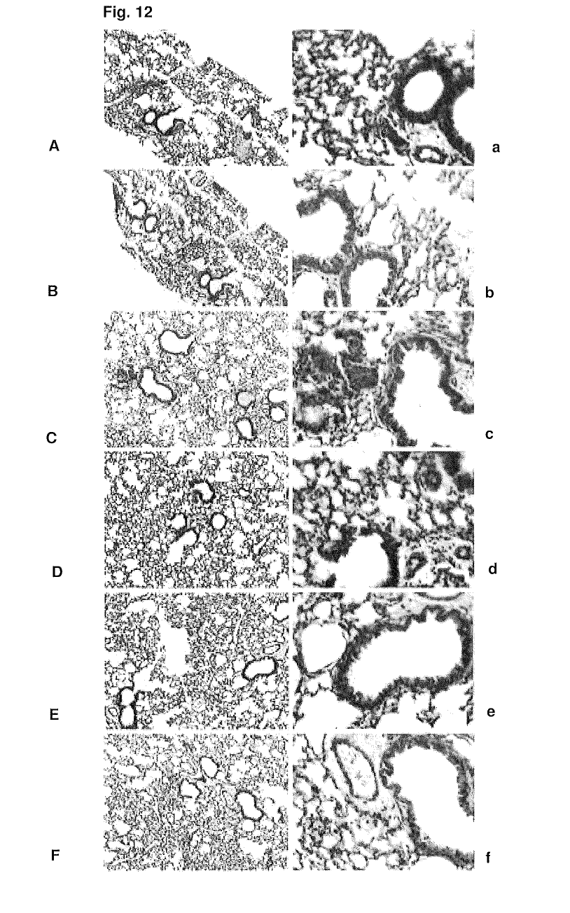

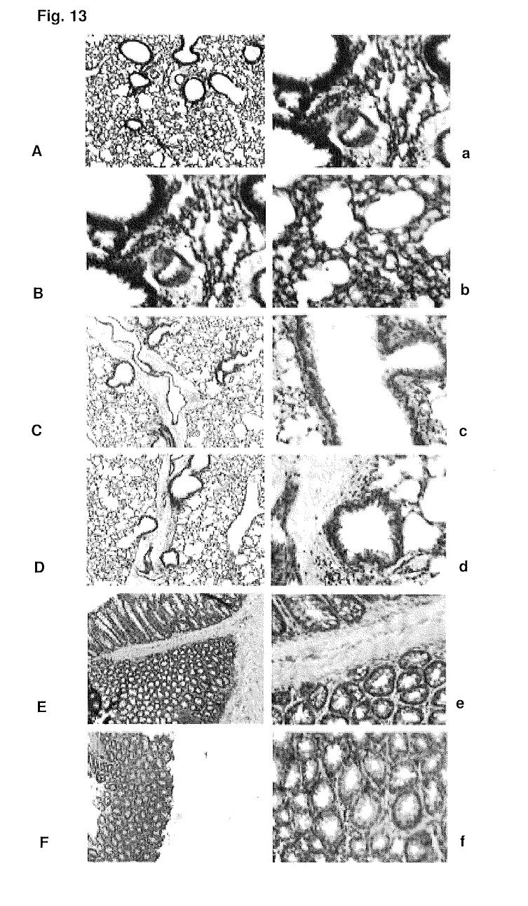

Neither extra-pulmonary effects nor localization was noted for EpCAM saline solution or INPs both on D8 and D19 (data not shown). Representative slides of EpCAM saline solution and INPs both on D8 and D19 are presented in FIGS. 5 and 6. As expected, specific localization of EpCAM within the lung bronchi is observed both for the EpCAM saline solution (FIG. 12 c, C) and EpCAM conjugated INPs (FIG. 12 e, E). However, the INPs were found to reside within the lung bronchi even on D19 (FIG. 13 c, C). The INPs are localized in association to the bronchi wall and not in pulmonary macrophages. It should be noted that the EpCAM expression is low within the lungs of healthy animals as depicted in FIG. 12 A,a. FIG. 13 e, E of the EpCAM marked expression within the mice colon is provided for validation purpose only. The signal observed is EpCAM specific as validated by the negative controls of sections exposed to secondary rat anti-mouse antibody solely (FIGS. 12 and 13 b, B, d, D, f, F).

### Discussion

This study was aimed to improve understanding of the safety and tolerability of pulmonary instilled NP and INP. The localization and the effect elicited by inhaled particles in the micrometer range is well-documented but extensive data are lacking for inhaled NPs. The pulmonary delivery of NPs opens up the possibility of enhanced, local therapeutic effects while reducing the dose and eventually the administration frequency. However, inhaled NPs may raise safety concerns. Toxicity data available so far include mainly the influence of inhaled non-therapeutic environmental particulate matter and it remains a debatable as to whether it may be extrapolated to drug loaded NPs. Furthermore, it was reported that the pulmonary effects elicited by inhaled NPs can depend on size and surface charge. Normally, the phagocytic cells residing in the lungs do not detect NPs in the size range of 120-150 nm, as in the present study. A major drawback of local particulate delivery to the lungs is the size-dependent elimination. Generally 80% of particles with a mass median aerodynamic diameter of less than 0.5 μM were found to be eliminated during exhalation. This size-dependent pitfall may be advantageous as NPs appear to settle effectively within the deep lungs by means of Brownian diffusion and present good pulmonary penetration.

Thus, it is important to follow the biofate of NPs following inhalation. PLA microspheres were observed to cluster in discrete groups in the lung tissue and were not evenly distributed². Nevertheless, this phenomenon was observed at the micrometer and not nanometer size range. Lai et al. studied recently the transport of PEGylated and nonPEGylated polymeric NPs in human mucus revealing that larger (500 and 200 nm NPs) PEGylated NPs as compared to smaller (100 mm NPs) non PEGylated NPs may transport quicldy in mucus that coats different organs including the cervicovaginal tract and the lungs. Stuart et al. investigated the interactions between gelatin NPs and artificial dipalmitoylphosphatidylcholine (DPPC) based lung surfactants using biophysical in vitro methods. Their results showed that interactions between the NPs and the lung surfactant film did not destabilize the monolayer thus suggesting that NP delivery is a possible and safe route of administration.

To evaluate the in vitro toxicity of the actual NPs, A549 cells were selected since this human lung adenocarcinoma cell line overexpresses the EpCAM epitope. Cell culture studies demonstrated that PEG-PLA NPs irrespective of their surface charge were devoid of cytotoxicity. The polymer dose tested in the present study (0.15 mg in 25 μl of 5% glucose solution) was selected on the basis of the toxicity results reported by other authors who investigated the pulmonary pro-inflammatory potential of DEAPA-PVAL-g-PLGA NPs in a single intratracheal instillation in a murine model using a dose range of 0.1-0.25 mg NPs in 100 μl of 5% glucose solution per mouse.

Similar PEG-PLA NP formulations with different surface charge properties elicited different toxicity profiles in vivo. Administration of cationic NPs resulted in increased mortality, reduced weight gain and elevated total BAL cells and macrophages specifically on D8. It should be emphasized that it was important to evaluate the influence of the surface charge of NPs, especially of cationic NPs on the lungs. Due to their positive charge, these NPs are expected to penetrate into the lung tissue following inhalation because of the electrostatic attraction with the lung cell membranes which carry a net negative charge due to the presence of proteins and phospholipid moieties. This should also allow efficient delivery of active ingredients to the external superficial layers of the alveoli and a prolonged residence time. Based on the safety and tolerability study, the most pronounced changes in hematological parameters were observed with this formulation, e.g. systemic leucopenia, elevated levels of neutrophils and stabs (shift to the left).

Decreased macrophage blood levels together with the elevated BAL macrophage values on D8 can imply on phagocytic cells recruitment to the lungs resulting in increased interaction of phagocytic cells and cationic NPs due to an electrostatic attraction. Overall these results may suggest pulmonary localization of cationic NPs the removal of which was mediated by macrophages up to the D8 whereas the INPs were still residing in the lungs even at D19. Anionic PEG-PLA NPs administration did not result in animal mortality and the overall appearance of mice was good although reduced weight was observed compared to the control on D19. The total BAL cell values were similar to that of the control saline with increased macrophage levels on D19. Changes in hematological parameters were moderate in comparison to the changes elicited by the cationic NP formulation. Pulmonary macrophage localization at D8 as well as congestion and inflammation processes returned to normal by D19. The overall toxic effects induced by anionic NPs appeared to be minor and reversible.

These encouraging data increase the chances for the broad use of anionic NPs for therapeutic and diagnostic pulmonary applications following pulmonary administration. Different MAbs are currently being tested for the treatment of various stages of lung cancer including MORAb-003, cetuximab, panitumumab and bevacizumab which is by far the most widely evaluated MAb in about 48 clinical trials for lung cancer treatment solely. In the current study, INPs were manufactured by conjugation of EpCAM MAb to PEG-PLA NPs based on experience gained from the conjugation of MAbs to cationic emulsions and NPs^{30,31} . . . . Specifically, EpCAM conjugated PEG-PLA NPs administration did not negatively affect the viability, appearance and weight of the mice. Total BAL cells did not increase initially and were similar to that of the control and the anionic PEG-PLA NPs. However, increased PMN and lymphocyte counts within the BAL were observed on D8. Furthermore, on D19 total cell number in BAL increased significantly. Specifically, BAL macrophage, lymphocyte and PMN populations were increased. Following the administration of EpCAM conjugated INPs increased blood stabs, monocytes and eosinphils values were observed on D8, suggesting initiation of an immune response. On D19 the changes were more moderate with increased hematological neutrophils and monocytes values.

Immunohistochemistry results of lung sections showed specific pulmonary localization of this formulation both on D8 and D19 which is specific to the bronchial wall and not macrophage related. It was recently suggested that EpCAM may promote cell proliferation and therefore, growth of the tumor. Thus, EpCAM conjugated INPs may be considered a potential drug delivery system following inhalation since the conjugated EpCAM is able to recognize and bind specifically to pathological lung tissues overexpressing EpCAM antigen as in the case of lung cancers. Histopathological assessment of the lungs indicated the presence of an inflammation reaction. It appears that this formulation differs from other formulations based on the elicited effects by D19. Since the effects of this formulation were different from the control antibody saline, it is possible that the INPs conjugate may have specific effects that resulted from the particulate delivery system per se causing pulmonary localization residence. It should be stressed that EpCAM Ab was from rat origin and thus could elicit species-related immune response as noted from the control Ab saline response (FIG. 9). It is therefore plausible that the increased BAL macrophages, PMNs and lymphocytes observed with the INPs can be attributed to the conjugation of the Ab to NPs that accentuated the immune response.

### Conclusions

Based on the results presented here, PEG-PLA NPs and EpCAM conjugated PEG-PLA NPs are pulmonary drug carriers for both local and targeted delivery following repeated NP administrations to the lungs. It appears that the positive surface charge of NPs resulted in increased pulmonary effects along with systemic toxicity and therefore is not recommended for local pulmonary administration. It does seem worthwhile to combine EpCAM-targeted therapy with selective anti-proliferative agents such as paclitaxel or vinoreline for lung cancer therapy. Furthermore, when the MAb EpCAM is conjugated to NPs; it mainly acts as a targeting moiety losing most of its own pharmacological and pharmacokinetic properties. Therefore, the effect of chemotherapy loaded EpCAM conjugated PEG-PLA NPs in in vitro and in vivo models of lung cancer is worthy of further evaluation.

Thus, within the context of the invention, preferably anionic nanoparticles (NPs having a negative surface charge) are used, in particular nanoparticles based on anionic PEG-PLA NPs.

TABLE 6
Hematological parameters of mice on the eighth day of experiment.

| | Control | | | Control | |
|---|---|---|---|---|---|
| | saline | Neg NPs | Pos NPs | EpCAM | EpCAM NPs |
| Parameter Units | n = 6 | n = 4 | n = 3 | n = 7 | n = 6 |
| WBC, 10³ cmm | 4.45 ± 0.494 | 4.5 ± 0.993 | 2.233 ± 0.406 | 4.343 ± 0.500 | 4.35 ± 0.640 |
| RBC, 10⁶ cmm | 10.34 ± 0.18 | 9.385 ± 0.428 | 10.163 ± 0.53 | 10.289 ± 0.14 | 9.77 ± 0.18 |
| HGB**, g/dl | 16.63 ± 0.29 | 14.58 ± 0.54 | 15.73 ± 0.80 | 16.66 ± 0.09 | 15.7 ± 0.28 |
| HCT, % | 51.93 ± 1.02 | 47.8 ± 2.52 | 54.93 ± 4.09 | 51.73 ± 0.73 | 48.61 ± 1.04 |
| MCV**, fL | 50.05 ± 0.34 | 50.85 ± 0.57 | 53.73 ± 1.94 | 50.06 ± 0.08 | 49.67 ± 0.15 |
| MCH, pg | 16.03 ± 0.14 | 15.55 ± 0.43 | 15.43 ± 0.07 | 16.13 ± 0.20 | 16.05 ± 0.11 |
| MCHC**, % | 32.05 ± 0.20 | 30.6 ± 0.97 | 28.8 ± 1.19 | 32.23 ± 0.41 | 32.32 ± 0.29 |
| PLAT*, 10³ cmm | 698 ± 39 | 582 ± 188 | 413 ± 123 | 918 ± 103 | 878 ± 50 |
| NEUT***, % | 17.83 ± 5.41 | 26.25 ± 2.50 | 23.33 ± 3.76 | 22.29 ± 1.85 | 21.33 ± 3.27 |
| STAB***, % | 0.0 ± 0.0 | 0 ± 0 | 0.67 ± 0.33 | 0 ± 0 | 0.17 ± 0.17 |
| LYMP***, % | 81 ± 5.56 | 72.75 ± 2.14 | 75.3 ± 3.48 | 76.71 ± 1.77 | 76.67 ± 3.51 |
| MONO***, % | 1 ± 0.37 | 0.75 ± 0.48 | 0.667 ± 0.67 | 0.57 ± 0.30 | 1.17 ± 0.31 |
| EOS***, % | 0.17 ± 0.17 | 0.25 ± 0.25 | 0 ± 0 | 0.43 ± 0.43 | 0.67 ± 0.21 |
| BASO, % | 0.0 ± 0.0 | 0 ± 0 | 0 ± 0 | 0 ± 0 | 0 ± 0 |

Results presented as mean ± SEM and the range of values below.
*p < 0.05;
**p < 0.01,
***p < 0.001
White blood cell, WBC; Red blood cell, RBC; Hemoglobin, HGB, Hematocrit, HCT; Mean corpuscular volume, MCV; Mean corpuscular hemoglobin, MCH; Mean corpuscular hemoglobin concentration, MCHC; Platelet, PLAT; Neutrophil, NEUT; Stabs, STAB; Lymphocyte, LYMP; Monocyte, MONO; Eosinophil, EOS; Basophil, BASO.

TABLE 7
Hematological parameters of mice on the nineteenth day of experiment.

| | Control | | | Control | EpCAM |
|---|---|---|---|---|---|
| Parameter | Saline | Neg NPs | Pos NPs | EpCAM | NPs |
| Units | n = 5 | n = 3 | n = 5 | n = 8 | n = 6 |
| WBC, 10³ cmm | 4.62 ± 0.29 | 4.30 ± 0.70 | 4.54 ± 0.60 | 4.59 ± 0.73 | 4.05 ± 0.35 |
| RBC**, 10⁶ cmm | 10.1 ± 0.14 | 10.31 ± 0.66 | 11.12 ± 0.3 | 9.66 ± 0.18 | 9.83 ± 0.16 |
| HGB, g/dl | 15.82 ± 0.17 | 16.6 ± 0.53 | 16.44 ± 0.17 | 16.39 ± 0.33 | 15.42 ± 0.38 |
| HCT***, % | 51.92 ± 0.79 | 56.6 ± 4.41 | 63.62 ± 4 | 48.46 ± 1.02 | 49 ± 0.79 |
| MCV***, fL | 51.26 ± 0.1 | 54.67 ± 1.01 | 56.78 ± 2.13 | 50.1 ± 0.13 | 49.8 ± 0.18 |
| MCH*, Pg | 15.62 ± 0.07 | 16.2 ± 1.10 | 14.78 ± 0.32 | 16.98 ± 0.43 | 15.65 ± 0.27 |
| MCHC***, % | 30.48 ± 0.17 | 29.63 ± 2.12 | 26.3 ± 1.53 | 33.9 ± 0.87 | 31.45 ± 0.50 |
| PLAT, 10³ cmm | 837 ± 26 | 549 ± 179 | 727 ± 937 | 566 ± 83 | 722 ± 60 |
| NEUT***, % | 21.4 ± 1.86 | 19 ± 4.04 | 24.6 ± 3.17 | 17.25 ± 3.58 | 28.5 ± 1.88 |
| STAB, % | 0 ± 0 | 0 ± 0 | 0 ± 0 | 0 ± 0 | 0 ± 0 |
| LYMP***, % | 77.6 ± 1.69 | 79 ± 3.51 | 73.8 ± 3.43 | 81.75 ± 3.80 | 69.83 ± 1.83 |
| MONO***, % | 0.6 ± 0.4 | 1.33 ± 0.7 | 1.2 ± 0.3 | 0.63 ± 0.32 | 1.33 ± 0.42 |
| EOS***, % | 0.4 ± 0.24 | 0.67 ± 0.33 | 0.4 ± 0.22 | 0.38 ± 0.18 | 0.33 ± 0.21 |
| BASO, % | 0 ± 0 | 0 ± 0 | 0 ± 0 | 0 ± 0 | 0 ± 0 |

Results presented as mean ± SEM and the range of values below.
*p < 0.05;
**p < 0.01,
***p < 0.001 denote significant differences between mean values measured in the indicated group.
White blood cell, WBC; Red blood cell, RBC; Hemoglobin, HGB, Hematocrit, HCT; Mean corpuscular volume, MCV; Mean corpuscular hemoglobin, MCH; Mean corpuscular hemoglobin concentration, MCHC; Platelet, PLAT; Neutrophil, NEUT; Stabs, STAB; Lymphocyte, LYMP; Monocyte, MONO; Eosinophil, EOS; Basophil, BASO.

### Pulmonary Delivery of Targeted Drug Loaded Immunonanoparticles as a Treatment for Non Small Cell Lung Cancer:

Objectives:

To examine the safety of pulmonary delivered INPs in a transgenic mouse model of non small cell lung cancer.

Methods:

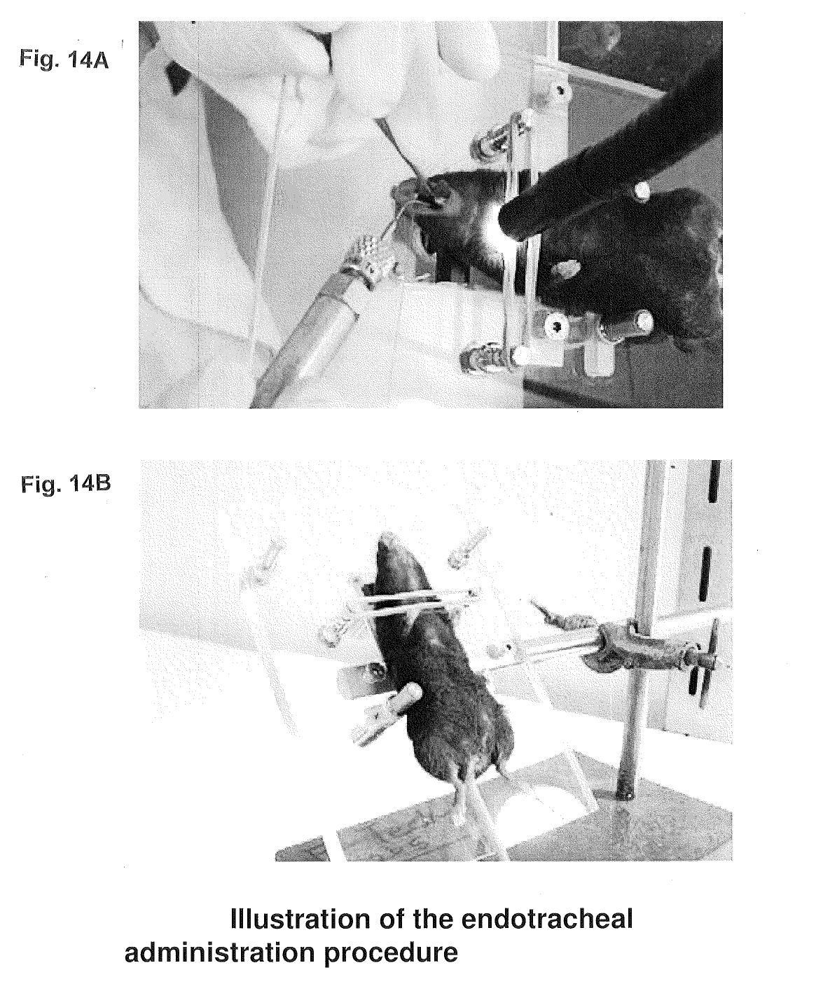

Pegylated polylactic acid) nanoparticles (NPs) loaded with paclitaxel palmitate (pcpl) were conjugated to an antibody (Ab) that recognizes the epithelial cell adhesion (EpCAM) molecule. Transgenic lung tumor bearing mice received formulations via an aerosol endotracheal administration (FIG. 14) over a 4 days period and were sacrificed on the 7th day. Treatments included blank (BLK) NPs (1.5 mg polymer), pcpl NPs diluted 1:5 with saline (0.3 mg polymer, 1 mg/kg pcpl) and EpCAM pcpl INPs diluted and undiluted with saline (0.3 mg polymer with 1 mg/kg pcpl or 1.5 mg polymer with 5 mg/kg pcpl, respectively). Body weight (BW), bronchial alveolar lavage (BAL), complete blood count, histochemistry and immunohistochemistry were used to assess both local and systemic effects.

### Results:

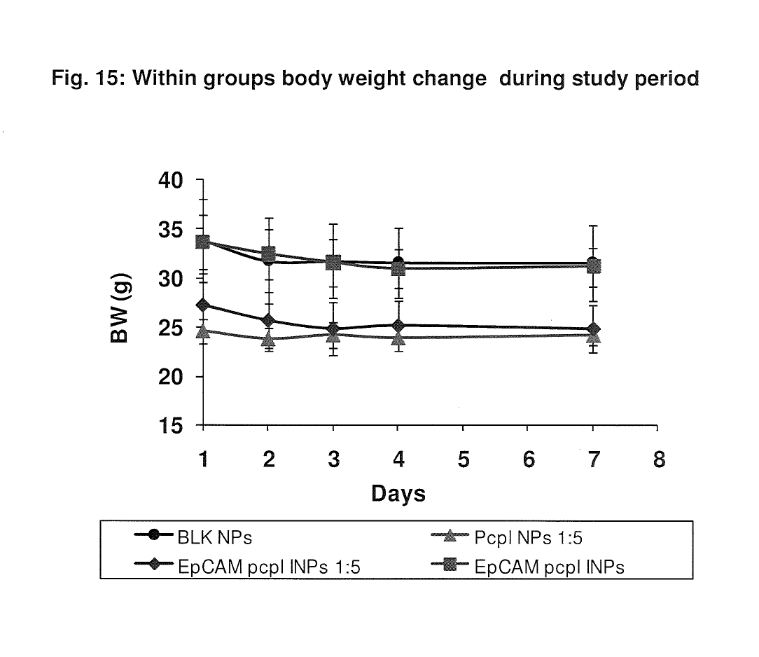

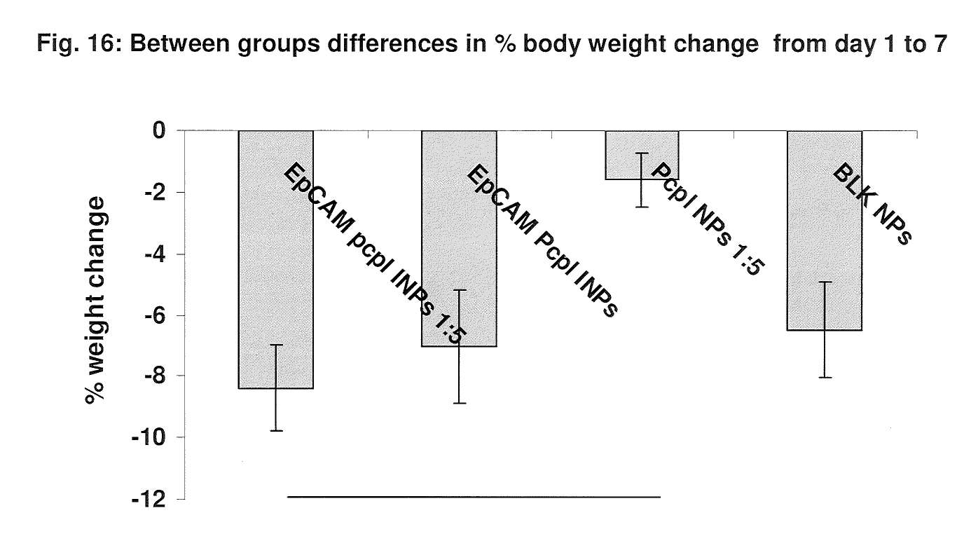

Within groups, animals' body weight was reduced by 8% at the most from day 1 to 7, a trend that was generally not statistically significant (FIG. 15). Within both BLK NPs and undiluted INPs, a similar weight loss was observed, suggesting there is no difference in the general well being of animals between the 2 groups. Between groups, only diluted INPs caused a statistically significant higher weight loss compared to diluted pcpl NPs (FIG. 16), possibly due to the lowest initial body weight of the latter (FIG. 15).

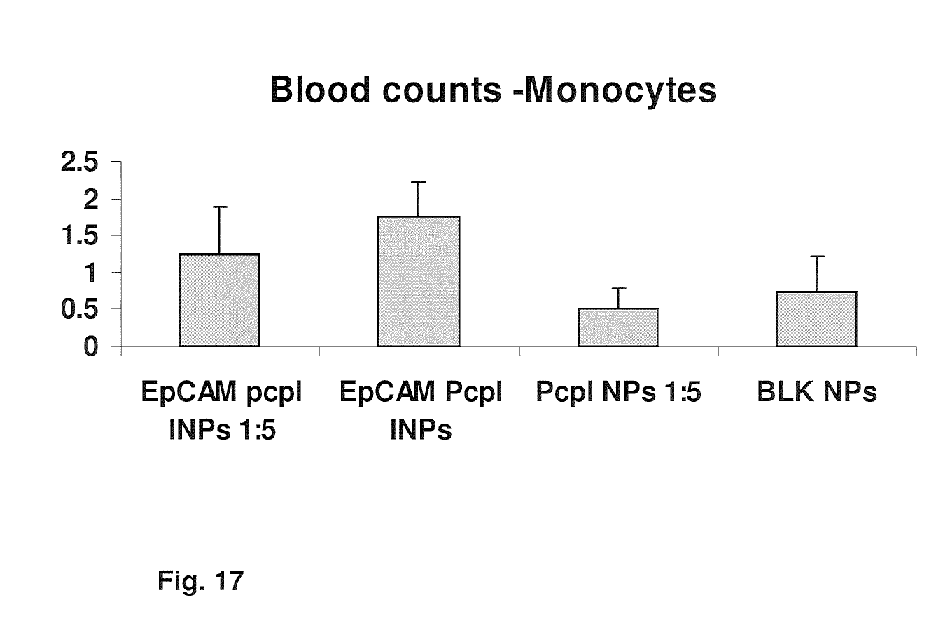

Increased monocytes count was also observed in the INPs treatments compared to untargeted BLK and Pcpl NPs, possibly due to an acute immune response to the rat antibody (FIG. 17).

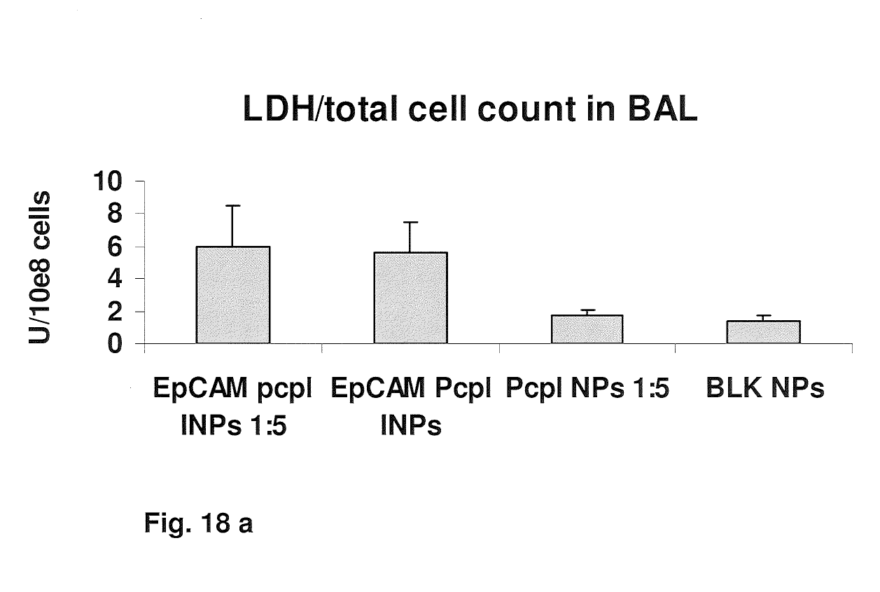

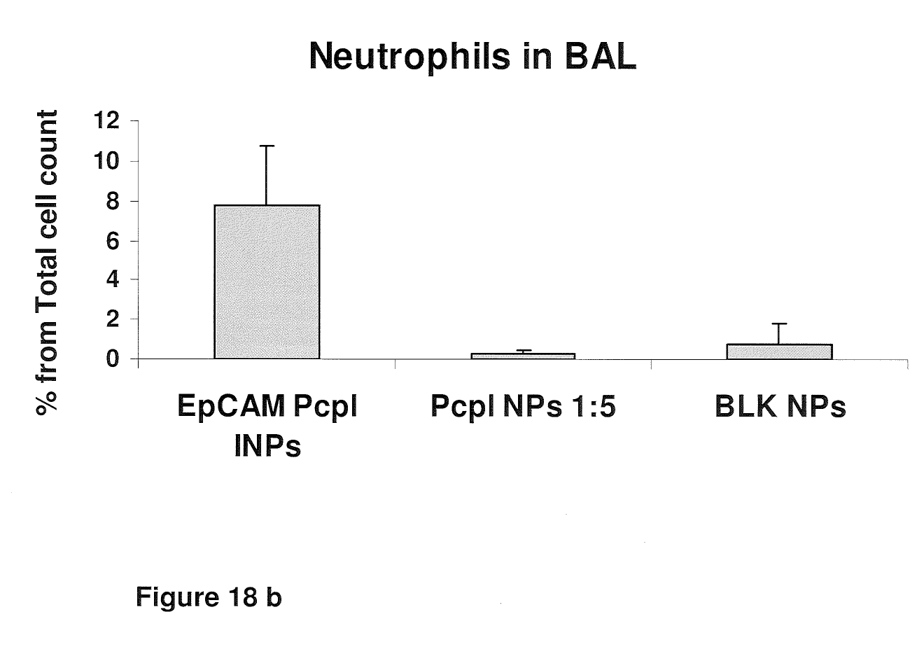

Bronchoalveolar lavage shows that pcpl-EpCAM INPs elicit greater local cell damage and inflammatory response than plain NPs, as reflected by higher LDH release (FIG. 18 a). This is due to a higher drug exposure and targeted tumor cells lysis. High local neutrophils counts are attributed to a rat Ab response (FIG. 18 b).

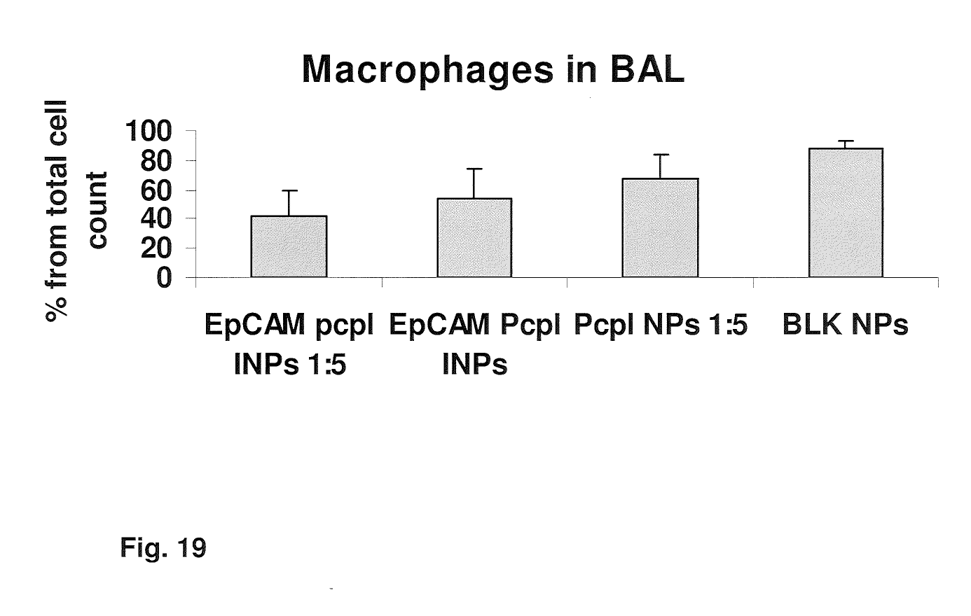

In contrast, plain NPs elicited a two fold higher macrophages recruitment than INPs (FIG. 19). This might indicate that plain NPs produce a higher nonspecific phagocyte response in the lung, while INPs manage to escape phagocytosis and decrease macrophages recruitment, thus inducing targeted cell damage.

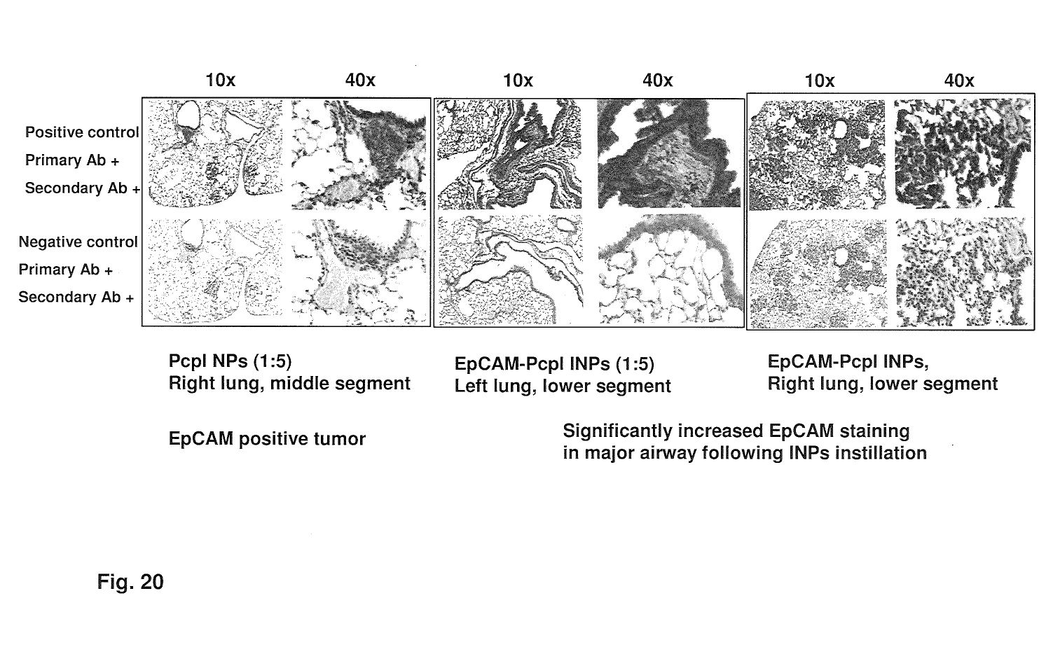

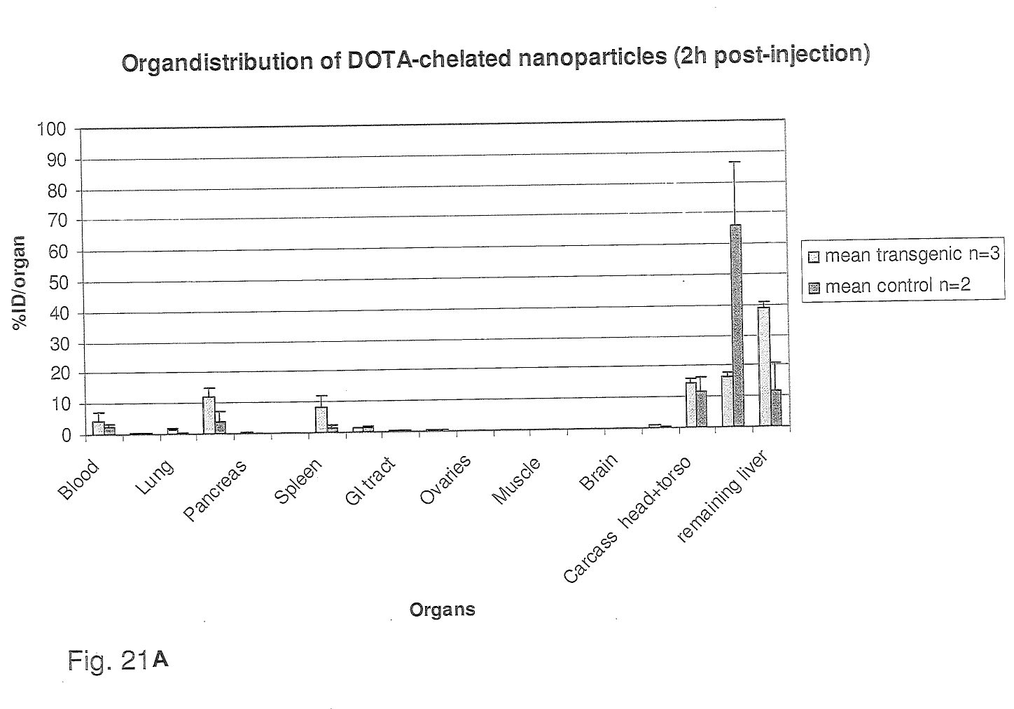

In addition, immunohistochemistry evidences EpCAM's presence in the lungs (FIG. 20) and provides evidence of lysis of tumor foci upon EpCAM INPs therapy.

### Conclusions:

Despite the relatively high concentration of administered polymer and animals' slight weight loss, all animals except one survived the four days of experiment until sacrifice on the 7th day. Altogether, the results show a safe and specific targeting of drug loaded INPs in the treatment of EpCAM positive lung cancer.

The advantages of coupling NPs containing lipophilic cytotoxic drugs with MAbs, preferably forming an aerosol comprising said delivery system, followed by local delivery to the lungs include: direct delivery to lungs, prolonged residence time in the target tissue; continuous release of significant potent drug doses at tumor sites and better cytotoxic drug internalization in tumors of the lung allowing improved efficacy. Respectively, the invention provides an enhanced system for direct delivery of radiopharmaceuticals and/or diagnostic agents to the lungs. The present invention concerns a delivery system administered to the lungs preferably by inhalation comprising a polymer-based nanoparticle; and a linker comprising a first portion non-covalently anchored to said nanoparticle, wherein at least part of said first portion comprises a hydrophobic/lipophilic segment embedded in said nanoparticle; and a second portion comprising a coupling group, preferably a maleimide compound, exposed at the outer surface of said nanoparticle. In accordance with one embodiment, the delivery system comprises one or more targeting agents, each covalently bound to said coupling group, preferably maleimide compound. In accordance with yet another embodiment, the delivery system comprises a drug and/or a radiopharmaceutical and/or a contrasting agent. A specific example for a linker in accordance with the invention is octadecyl-4-(maleimideomethyl)cyclohexane-carboxylic amide (OMCCA).

The features of the invention being disclosed in the preceding description, the subsequent drawings and claims can be of importance both singularly and in arbitrary combination for the implementation of the invention in its different embodiments.

1-20. (canceled)

21. A method of delivering an active agent to a target cell or target tissue affected by a lung disease or disorder, the method comprising comprises administering to a subject in need, an amount of a delivery system comprising:
i. a polymer-based nanoparticle;
ii. a linker comprising a first portion non-covalently anchored to said nanoparticle, wherein at least part of said first portion comprises a lipophilic segment embedded in said nanoparticle; and a second portion comprising a coupling group exposed at the outer surface of said nanoparticle to which at least one targeting agent, effective to target said delivery system to said target cell or target tissue, is covalently coupled; and
iii. said active agent.

22. The method of claim 21, comprising pulmonary administration of said delivery.

23. The method of claim 21, comprising administration of said delivery system by inhalation.

24. The method of claim 22, comprising delivery of said delivery system in the form of an aerosol.

25. The method of claim 21, wherein said at least one targeting agent comprises an antibody.

26. The method of claim 25, wherein said delivery system comprises at least two antibodies or antibody fragments, each with a different binding specificity.

27. The method of claim 21, wherein said at least one targeting agent is an antibody or an antibody fragment directed against an antigen selected from the group consisting of EP-CAM, VEGF, EGFR, HER2, HER3, HER4, CA 125, CTLA-4, H Ferritin.

28. The method of claim 21, wherein said at least one targeting agent comprises an antibody selected from the group consisting of Trastuzumab, Cetuximab, Bevacizumab, Panitumumab, Matuzumab, Nimotuzumab, MDX-447, Oegovomab, Pertuzumab, Ipilimumab, AMB8LK, anti mouse EpCAM, anti human-EpCAM.

29. The method of claim 21, wherein said lung disease or disorder is selected from lung cancer and bronchial dysplasia.

30. The method of claim 21, wherein said active agent is selected from the group consisting of a drug, a radiopharmaceutical agent, a contrasting agent and any mixture of same.

31. The method of claim 30, wherein said active agent is a drug selected from the group consisting of chemotherapeutic drugs and chemopreventive drugs.

32. The method of claim 31, wherein said drug is selected from the group consisting of docetaxel and paclitaxel palmitate.

33. The method of claim 30, wherein said active agent is a contrasting agent and said method is for imaging in the subject a target cell or target tissue affected by the lung disease or disorder.

34. The method of claim 21, wherein the nanoparticle is based on pegylated poly(lactide acid); the linker is octadecyl-4-(maleimideomethyl)cyclohexane-carboxylic (OMCCA) to which a monoclonal anti EP-CAM antibody is coupled, and the drug is palmitate Paclitaxel, said delivery system is in a form suitable for administration by inhalation.

35. A method of delivering a drug to a target cell or target tissue affected by a lung disease or disorder, the method comprises administering, via the respiratory tract of a subject in need of lung disease or disorder treatment or prevention, an amount of a delivery system comprising:
i. a polymer-based nanoparticle;
ii. a linker comprising a first portion non-covalently anchored to said nanoparticle, wherein at least part of said first portion comprises a lipophilic segment embedded in said nanoparticle; and a second portion comprising a coupling group exposed at the outer surface of said nanoparticle to which at least one targeting agent, effective to target said delivery system to said target cell or target tissue, is covalently coupled; and
iii. the drug.

36. The method of claim 35, wherein said targeting agent is a native antibody or a genetically engineered antibody comprising at least one antibody or fragment of an antibody selected from the group consisting of Trastuzumab, Cetuximab, Bevacizumab, Panitumumab, Matuzumab, Nimotuzumab, MDX-447, Oegovomab, Pertuzumab, Ipilimumab, AMB8LK, anti mouse EpCAM and anti human-EpCAM.

37. The method of claim 35, wherein said active agent is a drug selected from the group consisting of a chemotherapeutic drug and a chemopreventive drug.

38. The method of claim 35, comprising administering said delivery system by inhalation.

39. A method of imaging a target cell or target tissue affected by a lung disease or disorder, the method comprises administering, via the respiratory tract of a subject in need of cell or tissue imaging, an amount of a delivery system comprising:
i. a polymer-based nanoparticle;
ii. a linker comprising a first portion non-covalently anchored to said nanoparticle, wherein at least part of said first portion comprises a lipophilic segment embedded in said nanoparticle; and a second portion comprising a coupling group exposed at the outer surface of said nanoparticle to which at least one targeting agent, effective to target said delivery system to said target cell or target tissue, is covalently coupled; and
iii. a contrasting agent.

40. The method of claim 39, comprising administering said delivery system by inhalation.
